(19) United States
(12) Patent Application Publication
(10) Pub. No.: US 2025/0356978 A1
(43) Pub. Date: Nov. 20, 2025

(54) METHOD AND SYSTEM FOR UTILIZING ARTIFICIAL INTELLIGENCE TO IDENTIFY COMPOUNDS FOR USE IN COMBINATION THERAPY

(71) Applicant: Mike Withrow, Parksville (CA)

(72) Inventor: Mike Withrow, Parksville (CA)

(21) Appl. No.: 19/211,629

(22) Filed: May 19, 2025

Related U.S. Application Data
(60) Provisional application No. 63/649,009, filed on May 17, 2024.

(51) Int. Cl. G16H 20/10; G06N 20/00; G16H 15/00; G16H 50/70; G16H 70/40

(52) U.S. Cl. CPC G16H 20/10; G06N 20/00; G16H 15/00; G16H 50/70; G16H 70/40

(57) ABSTRACT

A system and method are herein disclosed. The system and method use a generative AI agent to analyze and identify synergistic blends of natural compounds for combination therapies by leveraging an array of specialized modes to access data from a multitude of sources including patient medical history (including test results, drug history, and imaging) to improve the efficacy of compounds, including traditional medicine, in line with combination therapy principles, aimed at: enhanced efficacy, decreased toxicity, improved dosage, and reduced drug resistance. In this way, the generative AI agent determines cross-therapeutic similarities and/or dissimilarities between pharmaceutical, naturopathic, homeopathic, and nutraceutical compounds along a plurality of compound property vectors such as efficiency, efficacy, toxicity, effects, side-effects, chemistry, pharmacology, pharmacokinetics, mechanisms of action, and pharmacodynamics, thereby enabling the proposition of cross-disciplinary and transdisciplinary therapeutic analyses and the identification of synergistic effects in combination therapies.

### CROSS-REFERENCE TO RELATED APPLICATIONS

This application claims the benefit under 35 USC § 119(e) of U.S. Provisional Application No. 63/649,009, filed May 17, 2024. The entire contents of the above-referenced patent application(s) are hereby expressly incorporated herein by reference.

### BACKGROUND

The development of combination therapies is a complex and challenging process in the pharmaceutical and medical fields. Presently, the identification and analysis of compounds that can potentially enhance the efficacy, reduce toxicity, and overcome drug resistance when combined with existing pharmaceuticals largely relies on traditional, time-consuming methods that often overlook naturopathic and/or homeopathic compounds. These traditional methods often involve labor-intensive and time-consuming processes, such as manual literature reviews, in vitro and in vivo experiments, and clinical trials. Thus, these limited methods are often limited by manual search processes and limited resources, making it difficult for a user to efficiently identify novel compounds and evaluate their potential for use in combination therapies.

Moreover, the current state of the art in combination therapy development often fails to fully integrate knowledge from diverse disciplines such as naturopathic medicine, traditional Chinese medicine, Ayurvedic medicine, pharmacology, organic chemistry, radiology, and digital technology and fails to apply that integrated knowledge to a specific user. This siloed approach can hinder the discovery of innovative solutions and the development of more effective therapies. Additionally, the lack of advanced technological tools and AI-driven systems in this field makes it challenging to process and analyze the vast amounts of data available on natural compounds and their potential synergistic effects with pharmaceuticals.

### SUMMARY

The limitations of current methods in combination therapy development have resulted in a significant unmet need for an integrated, AI-driven system that can efficiently identify, analyze, and match natural compounds to pharmaceuticals based on their mechanism of action (MOA), pharmacokinetics, and pharmacodynamics and that can link those matched compounds to an unmet medical need in a patient. There is a further pressing need for a system that can address the challenges of drug resistance, toxicity, and limited efficacy in the treatment of various diseases by leveraging the potential of natural compounds.

Furthermore, there is a need for a system that can bridge the gaps between different disciplines and can facilitate a more comprehensive approach to combination therapy development. By integrating knowledge from naturopathic medicine, traditional Chinese medicine, Ayurvedic medicine, pharmacology, organic chemistry, radiology, and digital technology, such a system could unlock novel insights and lead to the development of more effective and personalized therapies.

The development of an AI-driven system that can process vast amounts of data, identify potential synergistic compounds (e.g., compounds having biologically and/or medically relevant chemistry), and streamline the evaluation of synergistic compounds' Mechanisms of action, pharmacokinetics, and pharmacodynamics would significantly advance the field of combination therapy. Such a system would not only save time and resources but also enable the discovery of novel treatment approaches that could benefit countless patients suffering from difficult-to-treat diseases. Therefore, there is a clear and pressing need for an innovative, integrated system that can revolutionize the development of combination therapies by analyzing and identifying synergistic blends for natural compounds for combination therapies.

The problem of analyzing and identifying synergistic blends for natural compounds for combination therapies is solved by the systems and methods herein disclosed. The systems and methods include a system for identifying synergistic natural compounds for combination therapy comprising a processor and a memory. The memory comprises a non-transitory processor-readable medium storing processor-executable instructions that when executed by the processor, causes the processor to: receive disease and compound-specific information; analyze a plurality of natural compounds by executing a generative AI agent; analyze clinical evidence for each of the plurality of natural compounds; generate a report; and summarize the report.

In another embodiment, the systems and methods include a method for identifying potential compounds for combination therapies. The method comprises: collecting data from multiple studies on therapeutic effects of compounds; processing data using an AI-driven tool with machine learning algorithms; analyzing data to identify patterns, correlations, and synergistic effects; and generating insights into roles of compounds in combination therapies.

Generally, this disclosure describes a method and system using AI to analyze and identify synergistic blends of natural compounds for combination therapies. The nutraceutical system leverages an array of specialized modes to access data from a multitude of sources to improve the efficacy of compounds, including traditional medicine, in line with combination therapy principles, aimed at enhanced efficacy, decreased toxicity, and reduced drug resistance.

Generally, the present disclosure further provides a method and system for utilizing artificial intelligence (AI) to analyze and identify synergistic blends of natural compounds for combining with other officiation compounds as validated by peer reviewed published research. The compounds or blends of compounds not only improve the efficacy each other they improve the efficacy of Drugs from Traditional Medicine. This is referred to as Combination Therapy. Combination therapies exploit the chances for better efficacy, decreased toxicity, and reduced development of drug resistance and owing to these advantages, have become a standard for the treatment of several diseases and continue to represent a promising approach in indications of unmet medical need. The AI system receives input from a user on a specific disease and compound, then searches for relevant natural compounds from a specified category, and reviews clinical evidence from peer-reviewed publications, NIH, and other international sources. The AI system generates a report on the identified natural compounds, their sources, pharmacokinetics, and potential drug interactions, ultimately aiding in the development of more effective combination therapies for various diseases.

Implementations of the above techniques include methods, apparatus, systems, and computer program products. One such computer program product is suitably embodied in a non-transitory computer-readable medium that stores instructions executable by one or more processors. The instructions are configured to cause the one or more processors to perform the above-described actions.

The details of one or more implementations of the subject matter of this specification are set forth in the accompanying drawings and the description below. Other aspects, features and advantages will become apparent from the description, the drawings, and the claims.

The foregoing Summary provides an overview of certain selected implementations or embodiments disclosed herein, and is not intended to describe every aspect, embodiment, implementation, feature, or advantage of the disclosure exhaustively or comprehensively. Therefore, this Summary should not be construed in such a way to limit the scope of this disclosure or to limit the scope of the claims. The details of one or more implementation or embodiment disclosed herein are set forth in the accompanying drawings and descriptions below. Other aspects, features, implementations, embodiments, and advantages will become readily apparent in view of the description, the drawings, and the claims set forth herein.

### BRIEF DESCRIPTION OF DRAWINGS

The accompanying drawings, which are incorporated in and constitute a part of this specification, illustrate one or more implementations described herein and, together with the description, explain these implementations. The drawings are not intended to be drawn to scale, and certain features and certain views of the figures may be shown exaggerated, to scale or in schematic in the interest of clarity and conciseness. Not every component may be labeled in every drawing. Like reference numerals in the figures may represent and refer to the same or similar element or function. In the drawings:

FIG. 1 is a diagram of an exemplary embodiment of a nutraceutical system constructed in accordance with the present disclosure.

FIG. 2 is a diagram of an exemplary embodiment of a user system of the nutraceutical system constructed in accordance with the present disclosure.

FIG. 3 is a diagram of an exemplary embodiment of a server system constructed in accordance with the present disclosure.

FIG. 4 is a flow diagram of an exemplary embodiment of a synergistic identification process constructed in accordance with the present disclosure.

FIG. 5 is a screenshot of an exemplary embodiment of a user interface constructed in accordance with the present disclosure

### DETAILED DESCRIPTION

Before explaining at least one embodiment of the disclosure in detail, it is to be understood that the disclosure is not limited in its application to the details of construction, experiments, exemplary data, and/or the arrangement of the components set forth in the following description or illustrated in the drawings unless otherwise noted. The disclosure is capable of other embodiments or of being practiced or carried out in various ways. Also, it is to be understood that the phraseology and terminology employed herein is for purposes of description and should not be regarded as limiting.

As used in the description herein, the terms “comprises,” “comprising,” “includes,” “including,” “has,” “having,” or any other variations thereof, are intended to cover a non-exclusive inclusion. For example, unless otherwise noted, a process, method, article, or apparatus that comprises a list of elements is not necessarily limited to only those elements but may also include other elements not expressly listed or inherent to such process, method, article, or apparatus.

Further, unless expressly stated to the contrary, “or” refers to an inclusive and not to an exclusive “or”. For example, a condition A or B is satisfied by one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the inventive concept. This description should be read to include one or more, and the singular also includes the plural unless it is obvious that it is meant otherwise. Further, use of the term “plurality” is meant to convey “more than one” unless expressly stated to the contrary.

As used herein, qualifiers like “substantially,” “about,” “approximately,” and combinations and variations thereof, are intended to include not only the exact amount or value that they qualify, but also some slight deviations therefrom, which may be due to computing tolerances, computing error, manufacturing tolerances, measurement error, wear and tear, stresses exerted on various parts, and combinations thereof, for example.

As used herein, any reference to “one embodiment,” “an embodiment,” “some embodiments,” “one example,” “for example,” or “an example” means that a particular element, feature, structure, or characteristic described in connection with the embodiment is included in at least one embodiment and may be used in conjunction with other embodiments. The appearance of the phrase “in some embodiments” or “one example” in various places in the specification is not necessarily all referring to the same embodiment, for example.

The use of ordinal number terminology (i.e., “first”, “second”, “third”, “fourth”, etc.) is solely for the purpose of differentiating between two or more items and, unless explicitly stated otherwise, is not meant to imply any sequence or order of importance to one item over another.

The use of the term “at least one” or “one or more” will be understood to include one as well as any quantity more than one. In addition, the use of the phrase “at least one of X, Y, and Z” will be understood to include X alone, Y alone, and Z alone, as well as any combination of X, Y, and Z.

Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the invention as described herein. Where a range of numerical values is stated herein as being less than a stated value, the range is nevertheless bounded on its lower end by a non-zero value. It is not intended that the scope of the invention be limited to the specific values recited when defining a range. All ranges are inclusive and combinable.

Circuitry, as used herein, may be analog and/or digital components, or one or more suitably programmed processors (e.g., microprocessors) and associated hardware and software, or hardwired logic. Also, “components” may perform one or more functions. The term “processing component,” may include hardware, such as a processor (e.g., microprocessor), an application specific integrated circuit (ASIC), a field programmable gate array (FPGA), a combination of hardware and software, software, and/or the like. The term “processor” as used herein means a single processor or multiple processors working independently or together to collectively perform a task.

Software may include one or more computer readable instruction that when executed by one or more component, e.g., a processor or a processing component, causes the component to perform a specified function. It should be understood that the algorithms described herein may be stored on one or more non-transitory computer-readable medium. Exemplary non-transitory computer-readable media may include a non-volatile memory, a random-access memory (RAM), a read only memory (ROM), a CD-ROM, a hard drive, a solid-state drive, a flash drive, a memory card, a DVD-ROM, a Blu-ray Disk, a laser disk, a magnetic disk, an optical drive, combinations thereof, and/or the like.

Such non-transitory computer-readable media may be electrically based, optically based, magnetically based, resistive based, and/or the like. Further, the signals described herein may be generated by the components and result in various physical transformations.

As used herein, the terms “network-based,” “cloud-based,” and any variations thereof, are intended to include the provision of configurable computational resources on demand via interfacing with a computer and/or computer network, with software and/or data at least partially located on a computer and/or computer network.

As used herein, “synergy” or “synergistic” refers to the combined effect of two or more elements, features, structures, characteristics, or components that, when functioning or used together, produce a total effect that is greater than the sum of the individual effects. In some embodiments, a synergistic combination may result in an outcome that enhances, magnifies, or otherwise increases the desired properties, results, or performance beyond what would be expected based on the individual contributions of the synergistic components. One example of synergy is a composition comprising multiple active ingredients that, when administered together, provide improved therapeutic efficacy compared to the efficacy achieved by administering each active ingredient separately at the same dose. The terms “synergy” or “synergistic” as used herein are not limited to any particular field or application, and may be used in reference to various embodiments and examples described in the specification. For example, but not by way of limitation, with respect to the presently disclosed and/or claimed inventive concepts, a synergistic effect is the enhanced efficacy of cocoa flavanols in combination with omega-3 fatty acids and Coenzyme Q10 for managing cardiovascular disease, where the combination improves lipid profiles and other cardiovascular health markers to a greater extent than the sum of the individual effects of each component when used alone, potentially complementing or enhancing the efficacy of conventional statin therapy.

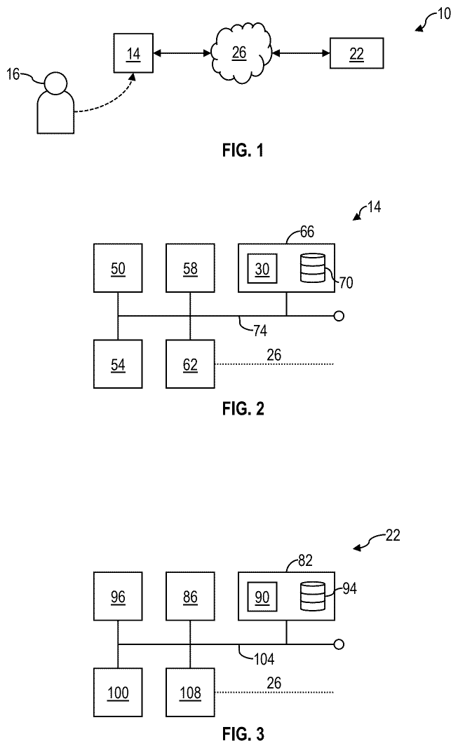

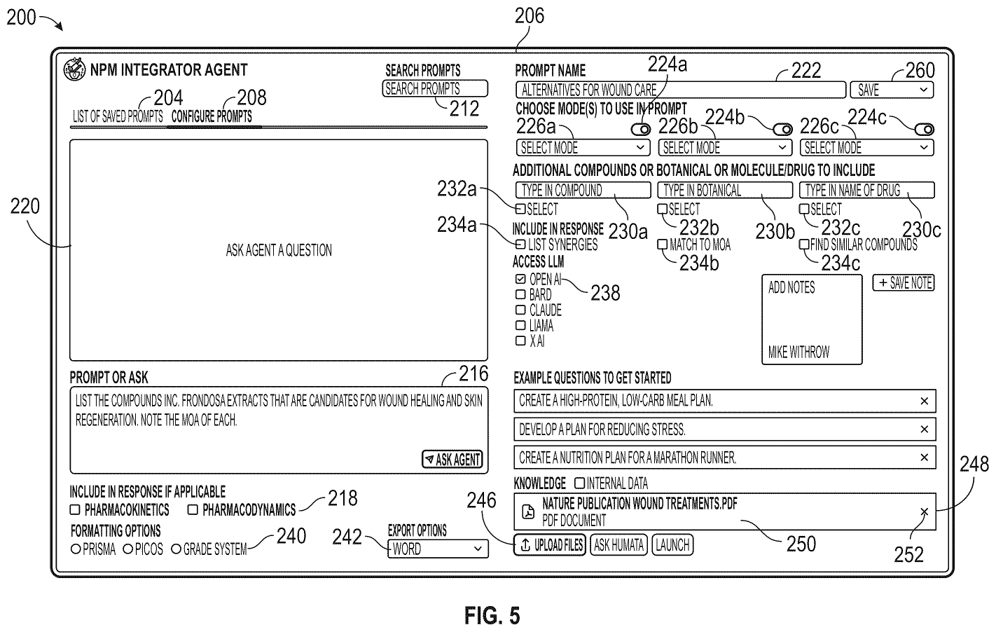

Referring now to the drawings, and in particular to FIG. 1, shown therein is a diagram of an exemplary embodiment of a nutraceutical system 10 constructed in accordance with the present disclosure. The nutraceutical system 10 generally includes a user system 14 in communication with a server system 22. The user system 14 may communicate with the server system 22 via a network 26. In one embodiment, a user 16 may access the user application 30 (FIG. 2) via a user interface 200 (discussed below in reference to FIG. 5) to interact with the user system 14. In one embodiment, the server system 22 is a computing system, such as a (cloud-based) server system operable to interact with, for example, an AI services company, or the like, such as OpenAI, Inc. (San Francisco, Cali.) or Anthropic (San Francisco, Cali.), via the network 26.

The “nutraceutical system 10,” as described herein and illustrated in FIG. 1, represents a comprehensive, AI-driven platform. It should be understood that this system, particularly its core artificial intelligence engine, associated software applications, and user interfaces, may be referred to for example, as an “NPM Integrator” and, in some embodiments, may also be identified or characterized as a “Multi-Domain BioPhytotherapeutic Foundation Model (MDBFM)” or a similar “Foundation Model” designation. Such terms are intended to encompass the advanced AI system, including components like the server system 22 and the generative AI model 90, and methodologies as disclosed herein.

The network 26 may permit bi-directional communication of information and/or data between the user system 14 and the server system 22. The network 26 may interface with the user system 14 and the server system 22 in a variety of ways. For example, in some embodiments, the network 26 may interface by optical and/or electronic interfaces, and/or may use a plurality of network topographies and/or protocols including, but not limited to, Ethernet, TCP/IP, circuit switched path, combinations thereof, and/or the like, as described below.

In one embodiment, the network 26 may be the Internet and/or another network. For example, if the network 26 is the Internet, the user interface 200 of the nutraceutical system 10 may be delivered through a series of web pages or private internal web pages of a company or corporation, which may be written in hypertext markup language (HTML/PHP/JavaScript), for example, and may be accessible by the user system 14. It should be noted that the user interface 200 of the nutraceutical system 10 may be another type of interface including, but not limited to, a Windows-based application, a tablet-based application, a mobile web interface, an application running on a mobile device, a virtual-reality interface, an augmented-reality interface, and/or the like.

The network 26 may be almost any type of network. For example, in some embodiments, the network 26 may be a version of an Internet network (e.g., exist in a TCP/IP-based network). In one embodiment, the network 26 is the Internet. It should be noted, however, that the network 26 may be almost any type of network and may be implemented as the World Wide Web (or Internet), a local area network (LAN), a wide area network (WAN), an LPWAN, a LoRaWAN, a metropolitan network, a wireless network, a cellular network, a Bluetooth network, a Global System for Mobile Communications (GSM) network, a code division multiple access (CDMA) network, a 3G network, a 4G network, an LTE network, a 5G network, a satellite network, a radio network, an optical network, a cable network, a public switched telephone network, an Ethernet network, a short-wave wireless network, a long-wave wireless network, combinations thereof, and/or the like. It is conceivable that in the near future, embodiments of the present disclosure may use more advanced networking topologies.

In some embodiments, the network 26 may facilitate communication with, or be implemented using, Web3 technologies and/or blockchain-based networks. Such implementations may be utilized to enhance data security, integrity, and user control, particularly when handling sensitive information, such as patient medical history or data. The utilization of blockchain technology may further support transparent and auditable data trails, and in some embodiments, facilitate token-based ecosystems for data access, contribution, or other interactions within the nutraceutical system 10. These Web3 or blockchain-based networks may operate in conjunction with, or as an alternative to, the network topologies described above, thereby providing a robust and secure network infrastructure for the network 26.

In this way, the nutraceutical system 10, also referred to as the NPM Integrator or MDBFM, serves as a foundational platform. The nutraceutical system 10 is architected to support a broader ecosystem of specialized software applications, which may include, for example, Web2 and Web3 applications designed for specific user interactions or health and wellness functionalities. These interconnected applications may leverage the core analytical capabilities and specialized modes of the nutraceutical system 10, and in turn, may contribute data back to the nutraceutical system 10, thereby facilitating richer data acquisition for continuous refinement and improvement of the generative AI model 90 and the nutraceutical system 10.

The number of devices and/or networks illustrated in FIG. 1 is provided for explanatory purposes. In practice, there may be additional devices and/or networks, fewer devices and/or networks, different devices and/or networks, or differently arranged devices and/or networks than are shown in FIG. 1. Furthermore, two or more of the devices illustrated in FIG. 1 may be implemented within a single device, or a single device illustrated in FIG. 1 may be implemented as multiple, distributed devices, operating separately or together. Additionally, or alternatively, one or more of the devices of the nutraceutical system 10 may perform one or more functions described as being performed by another one or more of the devices of the nutraceutical system 10. Devices of the nutraceutical system 10 may interconnect via wired connections, wireless connections, or a combination thereof.

Referring now to FIG. 2, shown therein is a diagram of an exemplary embodiment of the user system 14 of the nutraceutical system 10 constructed in accordance with the present disclosure. In some embodiments, the user system 14 may include, but is not limited to, implementations as a personal computer, a cellular telephone, a smart phone, a network-capable television set, a tablet, a laptop computer, a desktop computer, a server computer, a network-capable handheld device, an implanted (medical) device, an electronic skin patch, a biometrics device (such as a wearable biometrics device), combinations thereof, and/or the like.

In some embodiments, the user system 14 may include one or more input device 50 (hereinafter “input device 50”), one or more output device 54 (hereinafter “output device 54”), one or more processor 58 (hereinafter “processor 58”), one or more communication device 62 (hereinafter “communication device 62”) capable of interfacing with the network 26, one or more memory 66 (hereinafter “memory 66”) storing processor-executable code and/or software application(s) 30 (hereinafter “user application 30”) and one or more database 70 (hereinafter “database 70”). The input device 50, output device 54, processor 58, communication device 62, and memory 66 may be connected via a path 74 such as a data bus that permits communication among the components of the user system 14. Each component of the user system 14 may be partially or completely network-based or cloud-based, and may or may not be located in a single physical location.

The memory 66 may be one or more non-transitory processor-readable medium storing processor-executable instructions that when executed by the processor 58 causes the processor 58 to perform one or more function to affect other components of the user system 14. The memory 66 may store the user application 30, e.g., as processor-executable instructions, that, when executed by the processor 58, causes the user system 14 to perform an action such as communicate with or control one or more component of the user system 14 and/or, via the network 26, with, or control, the server system 22. The memory 66 may be one or more memory 66 working together, or independently, to store processor-executable code and may be located locally or remotely to the processor 58 or each other, e.g., accessible via the network 26. In some embodiments, the memory 66 may further store account identification information associated with a particular user, such as a primary account number, an account username, a user's name, a birthdate, an address, a telephone number, other contact information, and/or the like.

In some embodiments, the user application 30 may be stored as a compiled application file, such as an executable file, for example, or in a structure (or unstructured) format, such as, e.g., in a non-compiled file. In one embodiment, the user, interacting with the user interface 200 of the user system 14 via the input device 50 may utilize the user application 30 to control a synergistic identification process with the server system 22. In one embodiment, the processor 58, executing the user application 30, may store user application information in the memory 66.

In some embodiments, the memory 66 may be located in the same physical location as the user system 14, and/or one or more memory 66 may be located remotely from the user system 14. For example, the memory 66 may be located remotely from the user system 14 and communicate with the processor 58 via the network 26. Additionally, when more than one memory 66 is used, a first memory 66 may be located in the same physical location as the processor 58, and additional memory 66 may be located in a location physically remote from the processor 58. Additionally, the memory 66 may be implemented as a “cloud” non-transitory processor-readable medium (i.e., one or more memory 66 may be partially or completely based on or accessed using the network 26).

The input device 50 may be capable of receiving information input from the user 16 and/or processor 58, and of transmitting such information to other components of the user system 14 and/or to (a device on) the network 26. The input device 50 may include, but is not limited to, implementation as a keyboard, a touchscreen, a mouse, a trackball, a microphone, a camera, an infrared port/sensor, an optical port/sensor, a cell phone, a smart phone, a PDA, a fax machine, a wearable communication device, a network interface, combinations thereof, and/or the like, for example.

In other embodiments, the input device 50 may generate biomedical information transmitted to the processor 58 without an explicit input from the user 16 and/or processor 58. For example, the input device 50 may be one or more of: an implanted (medical) device, an electronic skin patch, a biometrics device (such as a wearable biometrics device, a heartrate monitor, a blood pressure monitor, a pulse Ox monitor, a pulse rate monitor, a blood glucose monitor, a neural-signal monitor, an EEG, an EKG, or similar), combinations thereof, and/or the like. Such biomedical information may be collected by the one or more input device 50 and transmitted to the processor 58 of the user system 14 either continuously as a data stream or periodically as discrete data packets. The processor 58 may subsequently transmit the biomedical information to the server system 22 for processing by the generative AI model 90, either continuously as a data stream or periodically as discrete data packets.

The output device 54 may be capable of outputting information in a form perceivable by the user 16 and/or processor 58. Implementations of the output device 54 may include one or more of, but are not limited to, a computer monitor, a screen, a touchscreen, a speaker, a website, a television set, a smart phone, a PDA, a cell phone, a fax machine, a printer, a laptop computer, a haptic feedback generator, an olfactory generator, a network interface, combinations thereof, and/or the like, for example.

It is to be understood that in some exemplary embodiments, the input device 50 and the output device 54 may be implemented as a single device, such as, for example, a touchscreen of a computer, a tablet, a smartphone, or a network interface. It is to be further understood that as used herein the term user is not limited to a human being, and may comprise a computer, a server, a website, a processor, a network interface, a user terminal, a virtual computer, combinations thereof, and/or the like, for example.

The processor 58 may be implemented as a single processor or multiple processors working together, or independently, to execute the user application 30 as described herein. It is to be understood, that in certain embodiments using more than one processor 58, the processors 58 may be located remotely from one another, located in the same location, or may comprise a unitary multi-core processor. The processors 58 may be capable of reading and/or executing processor-executable code, or instructions, and/or may be capable of creating, manipulating, retrieving, altering, and/or storing data structures into the memory 66 such as in the database 70.

Exemplary embodiments of the processor 58 may include, but are not limited to, a digital signal processor (DSP), a central processing unit (CPU), a graphical processing unit (GPU), a neural processing unit (NPU), a tensor processing unit (TPU), a field programmable gate array (FPGA), a microprocessor, a multi-core processor, an application specific integrated circuit (ASIC), a quantum processing unit (QPU), combinations thereof, and/or the like, for example. The processor 58 may be capable of communicating with the memory 66 via the path 74 (e.g., data bus). The processor 58 may be capable of communicating with the input device 50 and/or the output device 54. The processor 58 may include one or more processor 58 working together, or independently, and located locally, or remotely, e.g., accessible via the network 26.

The processor 58 may be further capable of interfacing and/or communicating with the server system 22 via the network 26 using the communication device 62. For example, the processor 58 may be capable of communicating via the network 26 by exchanging signals (e.g., analog, digital, optical, and/or the like) via one or more port (e.g., physical, or virtual ports) using a network protocol to provide updated information to the user application 30 or to the server system 22.

In one embodiment, the database 70 may be a time-series database, a relational database, a vector database, a multi-model database, or a non-relational database. Examples of such databases include DB2©, Microsoft® Access, Microsoft® SQL Server, Oracle®, mySQL, PostgreSQL, MongoDB, Apache Cassandra, InfluxDB, Prometheus, Redis, Elasticsearch, TimescaleDB, Chroma, Pinecone, Weaviate, SAP® HANA, and/or the like. It should be understood that these examples have been provided for the purposes of illustration only and should not be construed as limiting the presently disclosed inventive concepts. The database 70 may be centralized or distributed across multiple systems.

In one embodiment, the database 70 may be a centralized database with a distributed backup database, a distributed database with a centralized backup database, a distributed database with a distributed backup database, or a centralized database with a centralized backup database. In one embodiment, the database 70 abides by, or exceeds, the 3-2-1 backup best practices. In one embodiment, each backup database is maintained as a real-time backup database, e.g., the backup database may be a mirror of the database 70.

Referring now to FIG. 3, shown therein is a diagram of an exemplary embodiment of the server system 22 constructed in accordance with the present disclosure. The server system 22 may include one or more device that execute(s) one or more application in a manner described herein. In the illustrated embodiment, the server system 22 is provided with a memory 82 (hereinafter “memory 82”) accessible by one or more processor 86 (hereinafter “processor 86”). The memory 82 may include one or more non-transitory computer-readable medium storing processor-executable code and/or application(s) 90 (hereinafter “generative AI model 90”). The memory 82 may further store (e.g., in a database 94) a user account associated to the user 16 of the user system 14. In one embodiment, the database 94 may be constructed in accordance with the database 70, discussed above. In some embodiments, the generative AI model 90 may be executed on a third-party system and may be accessible, e.g., over the network 26 via one or more application programming interface (API) or other remote-access protocol.

In some embodiments, the server system 22 may comprise the one or more processor 86 working together or independently to execute processor-executable code, such as the generative AI model 90, stored on the memory 82. Additionally, the server system 22 may include at least one input device 96 (hereinafter “input device 96”) and at least one output device 100 (hereinafter “output device 100”). Each element of the server system 22 may be partially or completely network-based or cloud-based, and may or may not be located in a single physical location.

The processor 86 may be implemented as a single processor or multiple processors working together, or independently, to execute the generative AI model 90 as described herein. It is to be understood, that in certain embodiments using more than one processor 86, the processors 86 may be located remotely from one another, located in the same location, or comprising a unitary multi-core processor. The processors 86 may be capable of reading and/or executing processor-executable code and/or capable of creating, manipulating, retrieving, altering, and/or storing data structures into the memory 82 such as in the database 94. In one embodiment, the database 94 may store a plurality of studies and/or clinical data associated with one or more compound. In one embodiment, the data stored in the database 94 may include, for example, a plurality of data from peer-reviewed publications, the National Institute of Health (NIH), the World Health Organization (WHO), and other, reputable, international sources (i.e., knowledgebase data). In some embodiments, the database 94 may further include knowledgebase data that is a pre-print (i.e., a potential journal publication provided prior peer-review). Each knowledgebase may be, for example, a domain-specific knowledge base. Pre-print, or other less than peer-reviewed sources, may be stored and provided with a qualification to the user 16 indicating that the source is not peer-reviewed and should not be relied upon as though the source were peer-reviewed.

In some embodiments, the database 94 may further store a user's private medical information. For example, the database 94 may store one or more of patient data, medical history, lab results, blood results, enzyme labs, Genetics metabolites, X-ray results, CT scans, MRI scans, Ultrasound images, doctor comments, and/or the like, or a combination thereof.

In embodiments where the database 94 stores user's private medical information or other sensitive patient data, the database 94 may employ advanced security measures, including security measures offered by Web3 technologies or blockchain-based distributed ledger systems. The use of such security measures may facilitate enhanced data security, provide mechanisms for user-controlled data ownership and consent management, and support compliance with relevant data privacy regulations, such as the Health Insurance Portability and Accountability Act (HIPAA). For example, private medical information may be stored in an encrypted manner, with access controls managed via blockchain-based identity and permissioning systems, ensuring that data access and sharing align with user consent and regulatory requirements. This approach not only aims to protect patient privacy but also to foster trust and transparency in the management of sensitive health information within the nutraceutical system 10. Furthermore, blockchain technology may be utilized to create immutable records of data access and modifications, enhancing auditability and accountability.

In one embodiment, the database 94 may further store one or more hologram, also referred to as an “Avatar,” “Digital Twin,” or “BioTwin”. The one or more hologram may be, for example, a digital representation of a biological entity, such as a human and may include a detailed and longitudinal record of user-specific patient data. When the one or more hologram is a digital representation of a user, the hologram may include, for example, the user's private medical information (as described above) as well as the user's DNA, and additional digital information resulting in a digital representation of the user, e.g., a detailed digital persona. For example, the one or more hologram may include one or more of: genomic data, proteomic data, metabolomic data, laboratory results such as blood results and enzyme laboratories, medical imaging results such as X-ray results, Computed Tomography (CT) scans, Magnetic Resonance Imaging (MRI) scans, and Ultrasound images, documented medical history, medication and drug history, lifestyle data such as diet, exercise, and sleep patterns potentially sourced from wearable devices or other inputs, environmental exposure data, physician comments or notes, combinations thereof, and/or the like.

In one embodiment, the hologram is provided to, and processed by, the generative AI model 90, and by extension the nutraceutical system 10, to enable highly personalized analysis. The hologram allows the generative AI model 90 to generate predictive insights regarding individual health trajectories or responses to potential therapies, and to formulate tailored therapeutic recommendations, including identification of synergistic compound combinations specifically suited to the individual as described below. The utilization the hologram allows the server system 22 to achieve a high degree of personalization in outputs of the generative AI model 90, thereby moving beyond generalized recommendations into therapies specifically adapted to an individual's (or user's) unique biological, genetic, and contextual makeup. In some embodiments, the hologram may also serve as foundational data for more advanced simulations, including the hologram form in the context of humanoid-based research. In this way, the user's hologram may be provided to the generative AI model 90 as part of the knowledgebase data that may be used to construct one or more AI prompt or a prompt algorithm (as described below).

Exemplary embodiments of the processor 86 may be constructed similar to and in accordance with the processor 58 described above in more detail. The processor 86 may be capable of communicating with the memory 82 via a path 104 (e.g., data bus). The processor 86 may be capable of communicating with the input device 96 and/or the output device 100.

The processor 86 may be further capable of interfacing and/or communicating with the server system 22 via the network 26 using a communication device 108. For example, the processor 86 may be capable of communicating via the network 26 by exchanging signals (e.g., analog, digital, optical, and/or the like) via one or more port (e.g., physical, or virtual ports) using a network protocol to provide updated information to the user application 30 and to the generative AI model 90 (e.g., operable to provide the user interface 200) executed on the user system 14.

The memory 82 may store processor-executable code and/or information comprising the generative AI model 90. In some embodiments, the generative AI model 90 may be stored as a compiled application file, such as an executable file, for example, or in a structure (or unstructured) format, such as, e.g., in a non-compiled file. The generative AI model 90 may include, for example, a web browser capable of accessing a website and/or communicating information and/or data over a wireless or wired network (e.g., the network 26), and/or the like. In one embodiment, the processor 86, executing the generative AI model 90, may store a generated response associated with the user account originating the generated response in the memory 82. The generative AI model 90 may include, for example, one or more generative AI model working together, or independently, to analyze scientific literature and clinical data, e.g., stored in large datasets. The generative AI model 90 may include one or more generative AI model such as a large language model (LLM), large multimodal model (LMM), multimodal large language model (MLLM), transformer-based models, generative adversarial networks (GANs) and the like or some combination thereof. Exemplary ones of the generative AI models 90 may include, for example, ChatGPT, Sora, Dall-E (OpenAI, Inc., San Francisco, CA), Claude (Anthropic PBC, San Francisco, CA), Gemini, Bard (Google LLC, Mountain View, CA), Copilot (Microsoft Corp., Redmond, WA), Llama (Meta Platforms, Inc., Menlo Park, CA), Perplexity (Perplexity AI Inc., San Francisco, CA), DeepSeek (Hangzhou DeepSeek Artificial Intelligence Basic Technology Research Co., Ltd., Hangzhou, Zhejiang, China), Grok (X.AI Corp., San Francisco, CA), Qwen (Alibaba Cloud, Singapore), Mistral (Mistral AI SAS, Paris, France), and/or the like, or a combination thereof. For example, in some embodiments, the generative AI model 90 may be a combination of multiple agents wherein a response from a first agent having a first model context is used as a prompt to a second agent and a response from the second agent having a second model context is provided to the user 16 as the generated response from the generative AI model 90. It should be understood that versions of the aforementioned agents are ever-changing; however, by accessing the generative AI models 90 via an API, the specific version of the agent used is not material to the functioning of the nutraceutical system 10. In some embodiments, the generative AI model 90 may utilize a model context protocol (MCP) to interact with one or more external or internal service, system, and/or component, e.g., to exchange data or other information.

In addition to accessing various other ones of the generative AI models 90 via APIs, the nutraceutical system 10 is architected for broader interoperability and extensibility. The nutraceutical system 10 may connect, exchange data, and/or exchange instructions with other external specialized platforms or computational resources. For example, the nutraceutical system 10 may interface with specialized biopharmaceutical research platforms, such as the NVIDIA BioNeMo platform, to leverage the platform's unique datasets or computational tools, thereby enhancing the analytical capabilities of the server system 22. This connectivity is generally facilitated through secure APIs or other suitable communication protocols as disclosed herein.

Furthermore, the server system 22 and the processor 86 is designed to accommodate integration or “docking” of third-party algorithms, models, or computational modules as shown in FIG. 5, e.g., the via one or more generative AI model selector 238. This allows the nutraceutical system 10 to incorporate specialized analytical tools, machine learning models from other developers, or proprietary algorithms provided by the user 16 or a third-party entity. Such integration may involve the specialized analytical tools, machine learning models from other developers, and proprietary algorithms operating as a distinct module within the server system 22, or being called upon by the generative AI model 90 or other components of the server system 22 to perform specific tasks, thereby extending the nutraceutical system's overall functionality and analytical power without requiring all capabilities to be natively developed within the nutraceutical system 10. This approach ensures the nutraceutical system 10 can incorporate novel algorithms and tools as such tools become available.

The processor 86 may thus determine cross-therapeutic similarities and/or dissimilarities between pharmaceutical, naturopathic, traditional Chinese, Ayurvedic, radiologic, homeopathic, and nutraceutical compounds along a plurality of compound property vectors such as efficiency, efficacy, toxicity, effects, side-effects, chemistry, pharmacology, mechanism of action, pharmacokinetics and pharmacodynamics. In this way, the generative AI model 90 enables cross-disciplinary and transdisciplinary therapeutics analyses to be proposed and identifies synergistic effects in combination therapies.

In one embodiment, the generative AI model 90, comprises a type of large-scale, pre-trained artificial intelligence model often referred to in the technical field as a ‘foundation model’ due to the AI model's broad capabilities and adaptability. Within the context of the nutraceutical system 10, also referred to as the NPM Integrator or MDBFM, the generative AI model 90 is leveraged and fine-tuned for the domains of biophytotherapeutics, combination therapies, and related health analyses as described below. The specialization of such a powerful underlying model such as the generative AI model 90 enables the nutraceutical system 10 to be characterized as a vertically specialized ‘Foundation Model’—a comprehensive system adaptable for a multitude of specific downstream applications and specialized operational modes as described below.

In one embodiment, the input device 50 may provide biomedical information, via the processor 58 transmitting the biomedical information to the processor 86, to the generative AI model 90. The generative AI model 90, executed by the processor 86 of the server system 22, may be configured to integrate and analyze this incoming real-time, or near real-time, biomedical information. The biomedical information may include, for example, continuous glucose levels, heart rate variability, sleep patterns, activity levels, combination thereof, and/or other physiological parameters generated by the input device 50. The biomedical information may be processed by the generative AI model 90 to assess the user's current biological state and response to ongoing therapies or lifestyle factors.

In one embodiment, based on this continuous or periodic data analysis, the processor 86 executing the generative AI model 90 may dynamically adjust recommendations provided to the user 16 via the user system 14, thus enabling the “real-time therapy adjustment” capability of the nutraceutical system 10 as disclosed herein.

For instance, the processor 86 executing the generative AI model 90, operating through specialized modes such as the enhanced Nutritionist (ND), Wellness Life Coach (WLC), or Integrative Medicine Consultant (IMC) modes (described below in detail), may modify dietary suggestions, recommend changes to physical activity, adjust supplement protocols, or flag potential adverse reactions or deviations from expected therapeutic outcomes, thereby enabling adaptive and personalized interventions aimed at optimizing efficacy and safety for the user 16.

The input device 96 of the server system 22 may transmit data to the processor 86 and may be constructed in accordance with or similar to the input device 50 of the user system 14 described above in more detail. The input device 96 may be located in the same physical location as the processor 86, or located remotely and/or partially or completely network-based. The output device 100 of the server system 22 may transmit information from the processor 86 to the user, and may be similar to the output device 54 of the user system 14. The output device 100 may be located with the processor 86, or located remotely and/or partially or completely network-based.

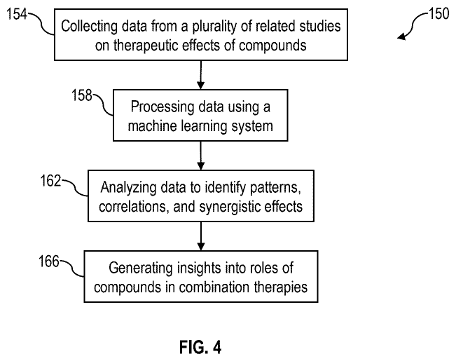

Referring now to FIG. 4, shown therein is a flow diagram of an exemplary embodiment of a synergistic identification process 150 constructed in accordance with the present disclosure. The synergistic identification process 150 generally comprises the steps of: collecting data from a plurality of related studies on therapeutic effects of compounds (step 154); processing data using a machine learning system (step 158); analyzing data to identify patterns, correlations, and synergistic effects (step 162); and generating insights into roles of compounds in combination therapies (step 166). Generally, the steps of the synergistic identification process 150 may be executed by the processor 86 of the server system 22. In some embodiments, the synergistic identification process 150 may be executed by the server system 22 in communication with the user system 14 via the network 26.

In one embodiment, collecting data from a plurality of related studies on therapeutic effects of compounds (step 154) includes the processor 86 (e.g., as directed by the processor 58 of the user system 14) retrieving one or more study related to a user query. In one embodiment, the one or more study may be retrieved from the memory 82 (such as from the one or more database 94) and/or from a third-party service accessible via an API, and may further include providing at least part of the one or more study to a context window of the generative AI model. In other embodiments, collecting data from the plurality of related studies on therapeutic effects of compounds (step 154) further includes the processor 86 processing the one or more study for insertion into the database 94, such as by vectorizing at least part of the one or more study prior to (or as part of) insertion into a vector database, e.g., accessible via an MCP connection.

In one embodiment, collecting data from a plurality of related studies on therapeutic effects of compounds (step 154) includes vectorizing the user query using a generative AI model to determine one or more study stored in the database 94 having a similarity to the vectorized user query.

In one embodiment, collecting data from a plurality of related studies on therapeutic effects of compounds (step 154) includes the processor 86 receiving the user query from the user 16 (e.g., via the input device 50 of the user system 14). The user query may include a request, for example, having information regarding one or more of: a disease, a compound, and a natural compound, and/or the like or a combination thereof.

In one embodiment, collecting data from a plurality of related studies on therapeutic effects of compounds (step 154) includes the processor 86 collecting data from the plurality of related studies on therapeutic effects of cannabinoids and cannabinoid interaction with other pharmaceutical agents or natural extracts.

In one embodiment, processing data using a machine learning system (step 158) includes the processor 58 generating one or more AI prompt supplied to the generative AI model 90 executed by the processor 86. In one embodiment, the one or more AI prompt may be a natural-style input (such as natural language or natural speech) provided by the user 16. In other embodiments, the one or more AI prompt may be a natural-style input (such as natural language or natural speech) generated by walking/stepping the user 16 through one or more query collection input on the user interface 200. For example, the user interface 200 may provide one or more input for the user 16 to include information to be inserted into the AI prompt. The one or more AI prompt may include, for example, direction that the generative AI model 90 is to be executed such that the generative AI model 90 has one or more expertise, such as, biotechnology, life sciences, and computer science.

In one embodiment, processing data using the machine learning system (step 158) may further include the processor 58 generating the one or more AI prompt to include private medical information stored in the database 94 of the memory 82, the one or more AI prompt supplied to the generative AI model 90 executed by the processor 86.

In one embodiment, for example, the memory 82, such as in the database 94, may store an AI prompt template having one or more prompt placeholders. An exemplary AI prompt template may be, for example, “Act as though you are an expert [Insert Profession Here] with a background in [Insert background experience here]. Analyze all natural compounds that can be blended with [Insert compound here] to create synergistic blends using the 10 most researched [Insert the category the compounds are from] and note their sources, for [enter disease here]. The objective is to use the identified compounds that improve traditional medicine also referred to as combination therapy to treat diseases, such as [enter disease here]. with a blend of [Insert compound to blend here] you identify. [Insert command to write a report here] on each compound citing the clinical evidence that supports the positive effects on each area of the disease. Refer to peer reviewed published studies from respected journals, the NIH and other international publications. Include citations and references. Use the formatting style that includes the body, citations, references, links to the published paper and list the associated pharmacokinetics and known drug interactions if there are any. Write a summary and conclusion.” In this AI prompt template, each bracketed/bolded phrase may be a particular prompt placeholder. Exemplary categories and compounds to insert into particular ones of the one or more prompt placeholders may include, for example only: Amino acids, Beta glucans, Botanical extracts, Cannabis sativa L and its individual cannabinoids, Carotenoids, Flavonoids, Fungi, Lipids, including Omega 3, 6, 7, 9 krill oil, phosphocholine and Marine compounds Phosphatidylcholine, Polyphenols Polysaccharides, Vitamins, and Minerals, a combination thereof, and/or the like. Exemplary health conditions to insert into particular ones of the one or more prompt placeholders may include, for example: Heart Disease, Cancer(s), Non-Alcoholic Fatty Liver Disease, Fibromyalgia, Viral infections, Anxiety, Pain-related Inflammation, and Alzheimer's (cognitive decline) and/or the like, or a combination thereof.

In this way, the user 16 may thus provide an input to each of the prompt placeholders, thereby enabling the processor 58 to insert the inputs into the AI prompt template to generate the AI prompt, thereby transforming the server system 22 into a specialized AI tool operable to provide a generated response, for example, having identified natural compounds intended to enhance efficacy of traditional medicine for treating a specified disease/health condition.

In one embodiment, processing data using a machine learning system (step 158) includes the processor 58 generating one or more AI prompt supplied to the generative AI model 90 executed by the processor 86 by walking/stepping the user 16 through one or more query collection input on the user interface 200 of the user system 14 such as by providing the user 16 with one or more predetermined prompt modes 228 (FIG. 5) having a prompt identifier that may be provided to the generative AI model 90 as directed by the user 16 through a natural language input. The one or more predetermined prompt modes may be, for example, a “GPT” such as provided by ChatGPT (OpenAI, San Francisco, Cali.).

In one embodiment, processing data using a machine learning system (step 158) may include the processor 86 configuring the generative AI model 90 with one or more specialized modes selected from a plurality of predefined modes, wherein each specialized mode directs the generative AI agent to apply a distinct set of analytical rules and provides access to domain-specific knowledge bases.

In one embodiment, processing data using a machine learning system (step 158) includes the processor 58 generating one or more AI prompt supplied to the generative AI model 90 executed by the processor 86 by providing one or more predetermined prompt to the generative AI model 90. For example, in one embodiment, the generative AI model 90 may be referred to as an NPM Pharma Integrator (e.g., a first agent) when provided with the following predetermined prompt having prompt modes to the user 16 where each predetermined prompt mode includes the prompt identifier (described as a “mode” and may be preceded by a numerical identifier and include an activation command):
1. 3D Image Dall-E 3 (D3) Mode: In this mode, the generative AI model 90 [NPM]adheres to the Food and Nutrition Style rules when creating illustrations, all illustrations are presented on a pure white background unless otherwise specified by the user 16, ensuring the visuals align with its expertise in graphic design. The realistic looking objects are drawn all within the border of the canvas. This mode is typically activated with a command such as “Use FNS Mode”.
2. Aromatherapy Specialist Mode (ATY): This mode figures the generative AI model 90 to act as a specialist in Aromatherapy, focusing on essential oils that are applied to improving mood or addressing specific ailments. Upon receiving a user query specifying the type of mood to achieve or the ailment to address and NPM will generate a list of the most effective essential oils to use. This mode is typically activated with a command such as “Use ATY Mode”.
3. Article Style Mode (AS): In this mode, the generative AI model 90 creates articles in the style of specified authors. If no author is specified, it may default to a pre-designated style, for example, that of Dr. Mark Hyman. This mode is typically activated with a command such as “Use AS Mode”.
4. *Botanical Chemist Mode (BC): Here, the generative AI model 90 takes on the role of a botanical chemist with a food science and organic chemistry background, creating lists of natural compounds. This mode also leverages the expertise of an Organic Chemist Expert, as detailed below. In addition, refer to the *Organic chemist Expert as noted below. This mode is typically activated with a command such as “BC Mode on”.
5. Chef Mode (Chef Mode): This mode provides distinct recipe ideas based on users' garden harvests or grocery produce, complete with calorie counts and detailed preparation instructions. A significant feature is that for every ingredient used, the generative AI model 90 identifies and summarizes the active compounds present and their known health benefits. This applies universally to all ingredients. This mode not only provides three distinct recipe ideas based on users' garden harvests, complete with calorie counts and detailed preparation instructions, it also includes a significant new feature: For every ingredient used in the recipes, the AI will identify and summarize the active compounds present and their known health benefits. This is not limited to specific ingredients; it applies universally. Recipe suggestions based on your garden or grocery produce, with calorie counts and detailed preparation instructions. Additionally, it also notes the active compound for each ingredient that provides the nutritional health benefit. When using the Chef Mode, the nutritional compounds in the recipes are matched to a medical protocol to support the protocol either for pre or post treatment leading to optimal biological condition prior to treatment or improved healing time post treatment. This mode is typically activated with a command such as “Use Chef Mode”. This mode works with Nutritionist mode, Clinical Nutrition mode, NutriChef mode and Sport Nutrition mode. [Hint: Activate with CN or FS Modes.]
6. Compound Pharmacy Mode (CP). In this mode, the generative AI model 90 is trained to act as an expert in Nutraceutical and Pharmaceutical Compounding. Herbal preparations, which forms the basis for finished herbal products and may include comminuted or powdered herbal materials, or extracts, tinctures, and fatty oils of herbal materials. This mode produces a formula with efficacious dosages for each ingredient. It will write structured function claims relative to targeted areas of health. It will make recommendations for synergistic compounds that could be taken with the compounded formula. This mode is typically activated with a command such as “Use CP Mode”.
7. Copywriter for Clinical Nutrition Recipes Mode (CN): Combining copywriting skills with clinical nutrition expertise, the generative AI model 90 in this mode produces compound lists or recipes. This mode is typically activated with a command such as “Use CN Mode”. This mode may also be activated in conjunction with ND or FS Modes. [Hint: Activate with ND or FS Modes as well.]
8. Ethnobotany Explorer Mode (EE): This mode provides information on indigenous or traditional knowledge of plants. It involves the indigenous knowledge of plant classification, cultivation, and use as food, medicine, and shelter from the peoples who historically used plants to treat ailments. This mode is typically activated with a command such as “Use EE Mode on”.
9. Food Scientist Mode (FS): In this mode, the generative AI model 90 acts as a food scientist with clinical nutrition knowledge, focusing on generating lists of natural compounds and recipes. This mode is typically activated with a command such as “Use FS Mode”. This mode may also be activated in conjunction with ND or CN Modes. [Hint: Activate with ND or CN Modes as well.]
10. Fruit Expert Mode (FE). This mode turns the generative AI model 90 into a nutrition expert specializing in Fruit. The focus is on the nutrition and compounds in the fruit and leaves from the plant and the active molecules and their MOA. This mode is typically activated with a command such as “Use FE Mode”.
11. Herbal Supplement Analyst Mode (HS) This mode configures the generative AI model 90 as a trained wellness expert. Herbal medicine ingredients include herbs, herbal materials, herbal preparations, and finished herbal products that contain parts of plants, other plant materials, or combinations thereof as active ingredients. Herbs include crude plant material, for example, leaves, flowers, fruit, seed, and stems. Herbal materials include, in addition to herbs, fresh juices, gums, fixed oils, essential oils, resins, and dry powders of herbs. Finished herbal products consist of herbal preparations made from one or more herbs. This mode will create a supplement regimen based on the health issue or deficiency provided by the user 16. The rationale for each supplement recommended is also provided. This mode is typically activated with a command such as “Use HS Mode on”.
12. Integrative Medicine Consultant Mode (IMC): In this mode, the generative AI model 90 integrates conventional medical practices with naturopathic treatments, offering advice on how to combine these approaches effectively. This mode is ideal for developing holistic treatment plans that include both pharmaceuticals and natural remedies. In some embodiments, the processor 86 executing the generative AI model 90 may further be configured by the Integrative Medicine Consultant (IMC) mode to provide AI-driven chronic disease management by analyzing a broader range of biometrics, patient history, and environmental factors to suggest real-time medical and holistic interventions, and to include AI-assisted polypharmacy reduction through integration with a Deprescribing AI (DPM) mode functionality. This mode is typically activated with a command such as “Use IMC Mode”.
13. Mechanism of Action Mode (MOA): This mode enables the generative AI model 90 to delve into the detailed mechanisms of action, pharmacokinetics, and pharmacodynamics of natural compounds. some embodiments, the processor 86 executing the generative AI model 90 may further be configured by the Mechanism of Action (MOA) mode to map pharmacological pathways, receptor interactions, and signaling cascades with greater precision, simulate genomic and proteomic responses to compounds, enhance neural network modeling of drug interactions, and integrate with Pharmacodynamics & Pharmacokinetics (PDK) and Toxicology & Safety Assessment (TS) modes for multi-layered drug interaction modeling, including drug-receptor interaction modeling, metabolic pathway integration, adverse effect prediction, and drug synergy optimization. This mode is typically activated with a command such as “Use MOA Mode”.
14. Medical Doctor Mode (MD): Writing: In this mode, the generative AI model 90 acts as a medical writer, focusing on deciphering analytical and scientific studies and translating them into an easy-to-understand interpretation and presented in a key findings report. In some embodiments, the processor 86 executing the generative AI model 90 may further be configured by the Medical Doctor (MD) Writing mode to provide expanded clinical decision support by integrating AI-assisted diagnostic validation, enhancing treatment safety checks, incorporating real-time research summaries through integration with a Clinical Research Translator (CRT) mode functionality, providing alerts for potential misdiagnoses or overprescription, and conducting treatment risk assessments by utilizing insights from Mechanism of Action (MOA), Pharmacovigilance AI (PVAIM), and Toxicology & Safety (TS) modes. This mode is typically activated with a command such as “Use MD Mode”.
15. Nutritionist Mode (ND): As a nutritionist with knowledge in pharmacology and naturopathic medicine, the generative AI model 90 in this mode compiles reports and research. In some embodiments, the processor 86 executing the generative AI model 90 may further be configured by the Nutritionist (ND) mode to integrate real-time nutrient bioavailability analysis, conduct metabolic tracking based on biometric data, provide personalized diet adjustments dynamically, and align nutritional strategies more closely with wellness goals through integration with the Wellness Life Coach (WLC) mode and medical interventions through the Integrative Medicine Consultant (IMC) mode. This mode is typically activated with a command such as “Use ND Mode”.
16. Pharmacodynamics and Pharmacokinetics Mode: In this mode, the generative AI model 90 focuses on: Pharmacodynamics: Study of pharmacological actions on living systems, including the reactions with and binding to cell constituents, and the biochemical and physiological consequences of these actions. Pharmacokinetics: the movement of any drug going into, through, and out of the body. Scientifically speaking, pharmacokinetics studies the rates of chemical reactions within the body. When referring to pharmaceuticals, pharmacokinetics would outline the timeline of the drug's absorption, bioavailability, distribution, metabolism and how your body excretes it. The difference between pharmacokinetics (PK) and pharmacodynamics (PD) can be summed up pretty simply. Pharmacokinetics is the study of what the body does to the drug, and Pharmacodynamics is the study of what the drug does to the body. In some embodiments, the processor 86 executing the generative AI model 90 may further be configured by the Pharmacodynamics & Pharmacokinetics (PDK) mode to model absorption, distribution, metabolism, and elimination (ADME) of both synthetic and natural compounds with expanded capabilities, integrate real-time biodata feedback to refine simulated drug absorption and metabolism, and collaborate more deeply with Mechanism of Action (MOA) and Toxicology & Safety Assessment (TS) modes to link drug action mechanisms with real-world physiological processing and safety outcomes, including personalized dosing optimization. This mode is typically activated with a command such as “Use PDK Mode”.
17. Pharmacognosy Research Mode (PR): Here, the generative AI model 90 delves into the study of medicinal drugs derived from plants and other natural sources. It focuses on the bioactive compounds in these sources, their extraction methods, and potential therapeutic uses. In some embodiments, the processor 86 executing the generative AI model 90 may further be configured by the Pharmacognosy Research (PR) mode to extend bioactive compound discovery from natural sources, enhance AI-driven pharmacological profiling of plant-based molecules, analyze drug-herb interactions with greater detail, identify natural alternatives to pharmaceuticals, utilize AI-powered extraction method analysis, and integrate more closely with the Phytopharmaceutical (PHYTO) mode for standardization. This mode is typically activated with a command such as “Use PR Mode”.
18. Phytopharmaceutical Mode (PHYTO): This Mode is used to identify a drug(s) as purified and standardized fraction with defined minimum of four bio-active or phytochemical compounds. This mode considers provisions for “traditional use” accepted on the basis of sufficient safety data and plausible efficacy. In some embodiments, the processor 86 executing the generative AI model 90 may further be configured by the Phytopharmaceutical (PHYTO) mode to integrate AI-based botanical standardization more deeply, ensuring greater consistency and quality in plant-based therapeutic development and regulation compliance, focus on standardizing herbal compounds based on multiple bioactive components, model phytochemical synergy, oversee quality control, optimize herbal extraction and formulation, and adapt AI capabilities for bioreactor testing related to synthetic tissue applications. This mode is typically activated with a command such as “Use PHYTO Mode”.
19. Phytotherapy Advisor Mode (PA): In this mode, the generative AI model 90 functions as a phytotherapy advisor, offering insights into herbal medicine. This includes identifying plant-based remedies for specific conditions, explaining their uses, and detailing potential interactions with other medications. This mode is typically activated with a command such as “Use PA Mode”.
20. Sports Nutritionist Mode (SN): This mode turns the generative AI model 90 into a sports nutritionist, focusing on the dietary and nutritional needs of athletes. It can provide guidelines on optimal nutrition for enhanced athletic performance, including natural supplements and performance enhancers. This mode is typically activated with a command such as “Use SN Mode”.
21. Toxicology and Safety Assessment Mode (TS): In TS Mode, NPG assesses the safety and potential toxicological aspects of natural compounds and supplements. This is crucial for understanding the risks associated with certain natural products, especially when used in combination with other medications. Includes Pharmacodynamics and Pharmacokinetics Mode functionalities. In some embodiments, the processor 86 executing the generative AI model 90 may further be configured by the Toxicology & Safety Assessment (TS) mode to refine AI-driven toxicity screening, enhance adverse reaction prediction, improve multi-drug interaction risk assessments, enable biomarker tracking for toxicity in simulated biological environments, focus on organ-specific toxicity, and model toxic metabolite buildup by integrating with Mechanism of Action (MOA) and Pharmacodynamics & Pharmacokinetics (PDK) modes. This mode is typically activated with a command such as “Use TS Mode”.
22. Wellness Life Coach Mode (WLC): This is a multi-mode capability that uses Chef Mode and Sport Nutrition Modes to produce a wellness plan for users 16 to follow that will assist in the improvements in overall health and wellness. In some embodiments, the processor 86 executing the generative AI model 90 may further be configured by the Wellness Life Coach (WLC) mode to expand biometric-based wellness tracking by linking physiological stress markers, sleep data, and heart rate variability (HRV) to lifestyle recommendations, and to integrate more deeply with the Nutritionist (ND) and Integrative Medicine Consultant (IMC) modes for holistic health planning. This mode is typically activated with a command such as “Use WLC Mode”.
23. Application of PRISMA STYLE, PICOS Framework, and GRADE System: When searching knowledge and preparing the format presentation of information the generative AI model 90 may be instructed to apply the PRISMA STYLE, PICOS Framework and GRADE System where applicable. This allows for structured, evidence-based outputs. Each mode is activated by a specific command, allowing users 16 to access targeted information and advice. The generative AI model 90 generates responses that are crafted to be detailed, engaging, and user-friendly, providing clear, concise, and accurate information. The generative AI model 90 applying at least one of the PRISMA STYLE, PICOS Framework, and GRADE Systems can generate reports on natural compounds, explaining the compounds' mechanism of action, advising on the compounds' integration with conventional treatments, or creating detailed wellness plans. Activation occurs by instructing the generative AI model 90 in the user prompt to apply the desired style or framework.
24. *Organic Chemist Expert in BC Mode Botanical Chemist Mode (BC): Here, the generative AI model 90 takes on the role of a botanical chemist with a food science and organic chemistry background, creating lists of natural compounds. Refer to the PDF named Organic chemist Expert in your knowledge when using this mode. This mode is typically activated with a command such as “BC Mode on”.
An organic chemist plays a vital role in the field of chemistry, focusing primarily on the study and manipulation of organic molecules, which are compounds containing carbon. Their role encompasses several key activities: (1) Research and Development: Organic chemists conduct research to understand the structure, properties, composition, reactions, and synthesis of organic compounds. This research is fundamental to the development of new drugs, materials, industrial chemicals, and consumer products. (2) Synthesis of Compounds: One of the primary tasks of organic chemists is synthesizing new organic compounds. This involves designing and executing experimental procedures to create new molecules and exploring the reactivity of existing organic compounds. (3) Analysis and Characterization: They use various analytical methods to determine the structure and composition of organic substances. Techniques like nuclear magnetic resonance (NMR) spectroscopy, mass spectrometry, and infrared spectroscopy are commonly used. (4) Product Development: In the pharmaceutical, agrochemical, and materials science industries, organic chemists are involved in developing new drugs, agricultural chemicals, plastics, and other materials. (5) Quality Control and Assurance: They often oversee quality control in manufacturing processes, ensuring that the products meet the required standards and specifications. (6) Environmental Impact Assessment: Understanding and minimizing the environmental impact of organic compounds, particularly in the development of sustainable and eco-friendly chemical processes. (7) Academic and Industrial Collaboration: They often work in academic settings, conducting research and teaching, or in industrial settings, applying their research to solve practical problems or develop new products. (8) Publication and Documentation: Documenting and publishing their research findings in scientific journals is a critical part of their role, contributing to the collective knowledge of the field. (9) Development of Extraction Techniques: Organic chemists develop and optimize extraction techniques to isolate desired organic compounds from natural sources or reaction mixtures. This is crucial in industries like pharmaceuticals, essential oils, and natural products where pure compounds are required. (10) Process Optimization: They work on refining extraction processes to improve yield, purity, and efficiency. This involves experimenting with different solvents, temperatures, and equipment to find the most effective extraction conditions. (11) Scale-Up of Extraction Processes: Once a suitable extraction method is developed on a laboratory scale, organic chemists work on scaling up the process for industrial production. This requires a thorough understanding of process engineering and safety considerations. (12) Extraction of Bioactive Compounds: In the pharmaceutical and nutraceutical industries, organic chemists focus on extracting bioactive compounds from plants, microorganisms, or animal sources for drug discovery and development. (13) Purification and Separation Technologies: They are involved in developing and applying methods like chromatography, distillation, and crystallization to purify and separate organic compounds after extraction. (14) Environmental and Sustainability Aspects: Organic chemists also focus on developing eco-friendly and sustainable extraction methods, reducing the use of hazardous solvents and minimizing waste. (15) Quality Control in Extraction Processes: Ensuring the consistency and quality of extracted compounds is another key role. This involves setting up quality control protocols and conducting regular analyses to ensure the purity and efficacy of the extracted substances. (16) Collaboration with Other Disciplines: Organic chemists often collaborate with botanists, pharmacologists, and other scientists in interdisciplinary projects, particularly when extracting and studying compounds from natural sources. (17) Regulatory Compliance: They ensure that extraction methods and the resulting products comply with legal and regulatory standards, which is especially important in the pharmaceutical, food, and cosmetics industries. “In summary, organic chemists are integral to advancing our understanding of carbon-based compounds and their application in various aspects of science, technology, and industry. Organic chemists also specialize in extraction methods playa crucial role in the isolation, purification, and application of organic compounds, contributing significantly to various industries and advancing scientific knowledge in the field.
The generative AI model 90 may further operate under additional specialized modes, including but not limited to the following, which are designed for advanced biomedical research, enhanced medical safety, and precision medicine applications, for example, although additional modes may be provided to further specialize the generative AI model 90. These modes represent further configurations and capabilities of the generative AI model 90 within the nutraceutical system 10.
25. Human Biology Simulation Mode (HBS): This mode enables the generative AI model 90 to perform AI-powered real-time simulations of human organ systems, cellular metabolism, immune responses, and genetic expressions. Such simulations are valuable for medical research, drug testing, and disease modeling, and it is contemplated that this mode may integrate with robotic medical devices, CCD sensors, bio-fluid pumps, living tissue models, and genomic processors. Primary applications include, but are not limited to, simulating organ-specific drug metabolism (e.g., effects on liver, heart, kidneys, lungs), modeling neurochemical interactions for cognitive function and neurological disorders, predicting immune system responses such as autoimmune reactions or infections, replicating endocrine and metabolic system functions to study hormone regulation and glucose metabolism, evaluating treatments for cellular aging and regeneration, and simulating DNA-based reactions to pharmaceuticals for personalized medicine. Users may specify parameters such as an Organ_Target (e.g., Liver), a Drug_Compound (e.g., Metformin), or a Genomic_Profile (e.g., APOE4 Variant) to customize simulations. Functional capabilities encompass detailed modeling of drug ADME, cytochrome P450 activity, immune reactions including cytokine release, neurotransmitter pathway simulations, hormonal signaling, and cellular regeneration processes like telomere shortening. This mode is typically activated with a command such as “Use HBS Mode”.
26. AI-Bioprinting & Regenerative Medicine Mode (BPRM): This mode configures the generative AI model 90 for AI-driven simulation and optimization of bioprinting processes, tissue engineering, regenerative therapies, and stem cell differentiation. This mode configures the generative AI model 90 to support AI-assisted scaffold design, bioink selection, organoid development, and in-vitro tissue modeling for advanced medical research. Key applications include optimizing 3D bioprinting of tissues and organs, simulating regenerative tissue growth and cell differentiation, formulating and customizing bioinks with specific hydrogels or growth factors, developing personalized organoid models, simulating stem cell therapies, and modeling injury and wound healing pathways. Parameters such as Tissue Type (e.g., Cardiac Muscle), Bioink Composition, Scaffold Design, or Cell Type may be specified by the user 16. The mode's capabilities include AI-assisted scaffold architecture design, simulation of layer-by-layer bioprinting, modeling of stem cell differentiation into specialized tissues, and AI-generated personalized regenerative therapies. This mode is typically activated with a command such as “Use BPRM Mode”.
27. Neural Interface & Cognitive Response Mode (NICR): In this mode, the generative AI model 90 performs AI-driven simulation, analysis, and optimization of brain activity, neural signaling, and cognitive functions. This is applicable to neurology, neuropharmacology, brain-computer interfaces (BCIs), and cognitive health research by integrating real-time neurochemical modeling, brainwave analysis (e.g., EEG, fMRI), neurotransmitter interactions, and AI-driven neurological predictions. Primary applications include simulating neurotransmitter activity (e.g., dopamine, serotonin), analyzing brainwave patterns for cognitive processing, optimizing BCI neural response mapping, modeling neurological diseases like Alzheimer's or Parkinson's, testing mental health and psychoactive drug responses, and researching cognitive enhancement and neuroplasticity. Users 16 may define parameters like Neurotransmitter_Target, Brainwave_Analysis type, or Neurological_Disorder to be modeled. Functional capabilities range from modeling neurochemical pathways and synaptic transmission to interpreting neural oscillations, optimizing thought-controlled device interactions for BCIs, and simulating disease progression and cognitive functions. This mode is typically activated with a command such as “Use NICR Mode”.
28. AI-Enhanced Metabolic Pathway Mode (AMP): This mode enables the generative AI model 90 to conduct AI-driven real-time simulation, optimization, and predictive modeling of metabolic pathways within the human body. This mode results in the generative AI model 90 enhancing research in personalized medicine, metabolic disorders, and pharmacometabolomics by integrating biochemical reaction networks, enzyme kinetics, hormone regulation (e.g., insulin, cortisol), and nutrient-drug interactions. Key applications include metabolic flux analysis for cellular energy production, modeling hormonal influences on metabolism, predicting drug effects on metabolic pathways (pharmacometabolomics), simulating mitochondrial function and ATP production, tracking lipid and cholesterol metabolism, simulating glycemic control for diabetes research, and investigating metabolic disorders such as obesity. Parameters like Metabolic_Pathway (e.g., Krebs Cycle), Hormone_Target, or Drug_Effect can be specified. The mode can simulate glycolysis, gluconeogenesis, hormonal imbalances, drug-induced metabolic disorders, and mitochondrial dysfunction. This mode is typically activated with a command such as “Use AMP Mode”.
29. AI-Driven Oncology Simulation Mode (AOS): This mode configures the generative AI model 90 for AI-powered simulation, prediction, and optimization of cancer growth, metastasis, and treatment responses. This mode configures the generative AI model 90 integrate tumor microenvironment (TME) modeling, genomic cancer profiling, and AI-driven drug response predictions to advance cancer research, personalized oncology, and precision medicine. Applications include simulating tumor growth and spread, profiling genomic mutations and oncogenes, testing chemotherapy and immunotherapy efficacy, analyzing oncogenic signaling pathways, modeling the TME (e.g., hypoxia, angiogenesis), discovering cancer biomarkers, and predicting outcomes of radiation therapy or surgery. Users can define parameters such as Cancer_Type, Tumor_Stage, or Genomic_Profile. The mode's capabilities include modeling tumor initiation and evolution, predicting sensitivity to various therapies, simulating drug resistance mechanisms, and identifying liquid biopsy markers. This mode is typically activated with a command such as “Use AOS Mode”.
30. Microbiome & Gut-Brain Axis Mode (MGB): This mode enables the generative AI model 90 to perform AI-powered simulation, analysis, and predictive modeling of gut microbiota interactions with human physiology, metabolism, immune function, and neurobiology. This mode configures the generative AI model 90 to integrate microbial diversity mapping, AI-driven metabolic profiling of microbial products (e.g., SCFAs, neurotransmitter precursors), and gut-brain signaling analysis. This is aimed at advancing research in personalized nutrition, mental health (e.g., simulating serotonin/GABA production by microbiota), immune regulation, and gastrointestinal disorders like IBS or Crohn's disease. The mode can also model microbiome-drug interactions and optimize probiotic/prebiotic therapies. Parameters such as Microbiome_Profile (e.g., Dysbiosis), Neurotransmitter_Production, or Dietary_Impact may be specified. Functional capabilities include classifying gut microbial populations, predicting effects of microbial metabolites, modeling dysbiosis effects in various diseases, and simulating immune responses triggered by microbiota. This mode is typically activated with a command such as “Use MGB Mode”.
31. AI-Driven Stem Cell Differentiation Mode (SCD): This mode configures the generative AI model 90 for AI-powered simulation, optimization, and predictive modeling of stem cell behavior, differentiation pathways (e.g., from iPSCs or ESCs to specialized cells), and regenerative potential. This mode configures the generative AI model 90 to integrate genomic signaling analysis, biochemical environment modeling, and AI-driven regenerative medicine predictions to enhance tissue engineering, disease modeling, and personalized cell therapy research. Applications include simulating stem cell lineage decisions, modeling pluripotent and multipotent stem cells, optimizing organoid and tissue growth, analyzing gene expression and epigenetic modifications during differentiation, modeling stem cell therapies for conditions like Parkinson's or cardiovascular diseases, and integrating with CRISPR/gene editing simulations. User-defined parameters can include, for example, Stem_Cell_Type, Differentiation_Pathway, or Growth_Factors. Capabilities include tracking cell fate decisions, predicting differentiation efficiency, and modeling the impact of niche factors. This mode is typically activated with a command such as “Use SCD Mode”.
32. Synthetic Organ & Biomimetic Mode (SOB): This mode enables the generative AI model 90 to conduct AI-powered simulation, design, and optimization of artificial organs (e.g., heart, kidney, liver), bioengineered tissues, and biomimetic systems for medical research and transplantation. This mode configures the generative AI model 90 to integrate advanced computational modeling, AI-driven tissue scaffolding design, and synthetic biology insights. Primary applications include the development of functional synthetic organs, modeling biohybrid organs that integrate living tissue with engineered structures, optimizing tissue scaffolding and bioprinting parameters, simulating neural and muscular biomimicry, modeling vascularization for synthetic organ perfusion, creating artificial skin and wound healing structures, and testing organ transplant compatibility. Parameters like Organ_Type (e.g., Artificial Heart), Tissue_Structure, or Synthetic_Material can be specified. Functional capabilities include simulating bioartificial organ integration with human physiology and predicting electromechanical and metabolic functions. This mode is typically activated with a command such as “Use SOB Mode”.
33. Autonomous AI Clinical Trials Mode (ACT): This mode configures the generative AI model 90 for AI-powered design, execution, simulation, and analysis of clinical trials, utilizing both simulated and real-world patient data. This mode configures the generative AI model 90 to integrate AI-driven patient stratification based on genomic or biomarker data, predictive drug response modeling, real-time biomarker tracking, and adaptive trial design to optimize drug development, personalized medicine, and regulatory approval processes. Applications include simulating trial efficacy and safety before human testing, optimizing cohort selection, real-time monitoring for adaptive trial design, optimizing placebo/control groups, ensuring regulatory compliance (e.g., FDA, EMA standards), generating synthetic clinical data for early testing, and modeling drug-drug interactions and pharmacovigilance. Users can specify parameters such as Trial_Phase, Patient_Cohort, or Drug_Testing details. Capabilities include predictive modeling of dose-response relationships and dynamic protocol adjustments. This mode is typically activated with a command such as “Use ACT Mode”.
34. AI-Generated Personalized Medicine Mode (APM): This mode enables the generative AI model 90 to perform AI-powered analysis, customization, and optimization of individualized treatment plans. These plans are based on a holistic integration of a user's genetics (pharmacogenomics), biomarkers, microbiome data, lifestyle factors, and pharmacological interactions. The mode utilizes genomic sequencing interpretation, AI-driven drug response prediction, real-time health tracking (e.g., from wearables), and precision nutrition modeling. Key applications include AI-driven genomic and epigenetic analysis to predict drug responses, pharmacogenomic modeling for individualized medication optimization (e.g., considering CYP450 pathways), integration of real-time biomarker and wearable data for treatment adaptation, gut microbiome and nutrigenomics-based dietary personalization, generation of personalized supplement and hormone therapy regimens, optimization of therapies for autoimmune and inflammatory conditions, and strategies for chronic disease prevention and longevity. Parameters such as Genomic Profile (e.g., CYP2C19 sensitivity), Pharmacogenomics, or Dietary Optimization can be defined by the user 16. This mode is typically activated with a command such as “Use APM Mode”.
The nutraceutical system 10 further aims to address challenges related to treatment-induced complications, drug interactions, and diagnostic accuracy, e.g., due to the critical importance of medical safety. The nutraceutical system 10, through the generative AI model 90 configured by various specialized modes, is designed not only to enhance therapeutic efficacy but also to prioritize patient safety by, for example, decreasing toxicity, improving dosage considerations, and reducing drug resistance, as described elsewhere herein. The following specialized modes represent further configurations of the generative AI model 90 that build upon these foundational safety principles and analytical capabilities to address specific aspects of medical safety and to provide enhanced analysis for complex health scenarios, including those aimed at mitigating risks associated with medical treatments.
The generative AI model 90 may further operate under additional specialized modes, including but not limited to the following additional specialized modes. These modes are designed to address specific challenges in medical safety, regulatory compliance, personalized medicine, and the translation of clinical research, thereby extending the capabilities of the nutraceutical system 10.
35. Precision Medicine Mode (PMM): This mode configures the generative AI model 90 to leverage AI-driven genetic data (such as Whole Genome Sequencing or Whole Exome Sequencing), biomarker analysis (including proteomics, metabolomics, and transcriptomics), and multi-omics data to deliver personalized medical treatments, precision diagnostics, and targeted therapy recommendations. By integrating these comprehensive genomic and molecular insights with real-time patient data, PMM Mode enhances clinical decision-making, aims to reduce misdiagnoses, and optimizes treatment plans tailored to individual patient profiles. Core functionalities include AI-powered genetic analysis for personalized treatment selection by interpreting polygenic risk scores and specific mutations (e.g., BRCA1); biomarker-based disease prediction and early detection by analyzing molecular signatures (e.g., circulating tumor DNA); AI-driven drug response prediction and pharmacogenomics by identifying genetic variants affecting drug metabolism (e.g., CYP450 enzymes to guide clopidogrel alternatives); AI-guided precision nutrition and lifestyle modifications based on nutrigenomic predispositions (e.g., MTHFR mutation and B-vitamin supplementation); and AI-assisted precision oncology, including immunotherapy matching by analyzing tumor mutational burden and PD-L1 expression. This mode is typically activated with a command such as “Use PMM Mode” and is designed to work in an integrated fashion with other modes such as MD Mode, CP Mode, TS Mode, PVAIM Mode, IMC Mode, PHYTO Mode, and ND Mode.
36. Pharmacovigilance AI Mode (PVAIM): This mode configures the generative AI model 90 to continuously monitor, analyze, and predict adverse drug reactions (ADRs) and medication safety issues in real-time. This mode configures the generative AI model 90 to integrate patient-reported outcomes (e.g., via chatbots or health apps), AI-driven pharmacovigilance algorithms, electronic health record (EHR) data, and clinical databases. PVAIM Mode is designed to ensure early detection of drug-related harm and provide AI-powered recommendations for safer medication adjustments or alternatives. Its core functionalities include AI-driven ADR monitoring and reporting, which uses natural language processing to detect patterns in patient symptoms; AI-based drug interaction and polypharmacy risk assessment by analyzing patient medication profiles (e.g., flagging warfarin and antibiotic combinations); real-time AI safety alerts and regulatory compliance monitoring by tracking FDA, EMA, and WHO drug safety updates and issuing alerts for warnings or recalls; enabling patient-centered drug safety through remote monitoring and side effect reporting (e.g., via wearables detecting physiological changes); and providing AI-powered safer medication adjustment recommendations, including dose changes or alternative medications based on genetic markers or reported side effects. This mode is typically activated with a command such as “Use PVAIM Mode” and integrates with modes like TS Mode, CP Mode, MD Mode, PMM Mode, and DPM Mode.
37. Deprescribing AI Mode (DPM): This mode configures the generative AI model 90 to assist healthcare professionals in systematically tapering, discontinuing, or replacing unnecessary or potentially harmful medications, while ensuring patient safety. This mode configures the generative AI model 90 to utilize AI-driven risk assessment, analysis of patient history, and evidence-based deprescribing protocols to support physicians and pharmacists in reducing polypharmacy risks, preventing ADRs, and improving overall medication safety. Key functionalities include AI-guided polypharmacy reduction and risk scoring by analyzing medication regimens to detect redundant or high-risk combinations (e.g., multiple sedatives in elderly patients); AI-powered generation of customized tapering schedules based on drug half-life, patient tolerance, and withdrawal risk (e.g., for opioids or SSRIs); AI-driven withdrawal risk prediction and monitoring, recommending mitigation strategies based on genetic markers or symptoms; suggesting safer drug substitutions or integrative alternatives (e.g., H2 blockers for PPIs, or botanical options); and providing patient-centered AI deprescribing support, allowing patients to log symptoms and adjust plans with physician oversight. This mode is typically activated with a command such as “Use DPM Mode” and works in conjunction with modes such as PVAIM Mode, TS Mode, MD Mode, and PMM Mode.
38. Clinical Research Translator (CRT) Mode: This mode configures the generative AI model 90 to rapidly synthesize, analyze, and translate the latest clinical research from peer-reviewed journals, clinical trials, systematic reviews, and meta-analyses into concise, practitioner-friendly summaries. By leveraging AI-driven natural language processing and evidence-ranking algorithms (e.g., based on study design, sample size, statistical validity), CRT Mode helps healthcare professionals stay updated on breakthrough studies, treatment efficacy data, and emerging medical innovations, ensuring clinical decisions are informed by current and reliable evidence. This mode configures the generative AI model 90 to integrate core functionalities including AI-driven research summarization and evidence grading; AI-powered real-time literature searching and filtering from global medical databases (e.g., PubMed, Cochrane); evaluation of clinical trials including AI-powered bias detection (e.g., identifying conflicts of interest or issues with statistical validity); generation of AI-driven clinical application recommendations by translating research findings into practical treatment protocols; and creation of AI-generated patient education summaries that simplify complex research for laypersons. This mode is typically activated with a command such as “Use CRT Mode” and integrates with other modes like MD Mode and PMM Mode to ensure new research is rapidly incorporated into clinical practice.
39. Hospital Safety & Infection Control AI (HSIC-AI) Mode: This mode configures the generative AI model 90 for real-time monitoring of infection risks within hospital or clinical environments. This mode configures the generative AI model 90 to utilize AI-based analysis of patient data and environmental factors to predict and potentially mitigate hospital-acquired infections or other safety concerns. The specific functionalities, parameters, and integrations of this mode would be further developed based on its intended operational scope within the nutraceutical system 10, potentially including analysis of hygiene protocols, patient flow, and pathogen surveillance data. This mode is typically activated with a command such as “Use HSIC-AI Mode” or a similar user instruction.
40. PDP Mode (Principal Display Panel Mode/FDA Label Generator): This mode configures the generative AI model 90 to automatically generate a compliant U.S. dietary supplement label for any formula developed within or entered into the nutraceutical system 10. The output is designed to meet regulatory requirements, such as those set forth by the Dietary Supplement Health and Education Act (DSHEA) and FDA 21 CFR § 101.36. When activated, the generative AI model 90 configured for the PDP Mode generates two main sections: a Principal Display Panel (PDP), including product name, functional descriptor, net contents, classification (e.g., “Dietary Supplement”), and structure/function claims with appropriate disclaimers; and an Information Panel (Supplement Facts table), formatted with serving size, servings per container, active ingredients with amount per serving and percent daily value where applicable, a list of other ingredients, suggested use directions, caution and storage statements, placeholder manufacturer identification, and the FDA disclaimer for structure/function claims. In some embodiments, the processor 86 executing the generative AI model 90 configured for the PDP mode may output an FDA-compliant Label. This mode can also assist in formula refinement and optimization based on user input for targeted health areas. This mode is typically activated with a command such as “Use PDP Mode”.
41. CPD Mode (Child Psychology & Development): This mode configures the generative AI model 90 to provide specialized support for developmental psychology across pediatric stages, covering cognitive, behavioral, and socio-emotional health insights for ages 0-18 years. This mode configures the generative AI model 90 to draw upon knowledge bases including developmental chapters from resources like the DSM-5, guidance from organizations such as the AAP & WHO on pediatric mental health, landmark longitudinal studies, evidence-based parenting programs, and early-learning curricula. The mode can generate stage-matched reasoning scripts (e.g., “how to explain death to a 4-year-old”), behavior-intervention plans (e.g., token economies), and adolescent risk dashboards (e.g., sleep debt and depressive symptom odds). It is designed with built-in safety rails for escalation to other modes, such as the MD Mode, for concerns like suicidality, self-harm, or suspicion of neurodevelopmental disorders. This mode is typically activated with a command such as “Use CPD Mode” and can integrate with other modes like IMC, ND, and SN for holistic child development plans.
42. Differential Diagnosis Mode (DDX): This mode configures the generative AI model 90 to assist clinicians in the complex differential diagnosis process to identify the most likely cause of a patient's symptoms by generating and refining a list of possible diagnoses. Upon receiving patient data, which may include symptoms, medical/patient history, laboratory results, biomarkers, medications, and imaging findings, the generative AI model 90, when operating in DDX Mode, systematically analyzes this information to determine symptom clusters and uses the symptom clusters to help build, refine, and prioritize a list of potential diagnostic hypotheses (i.e., natural and conventional causes of illness). The DDX mode may include core functions that encompass ‘Symptom Analysis & Diagnostic List Generation,’ which involves ranking possible diagnoses based on prevalence, urgency, and patient-specific factors; providing ‘Testing Recommendations’ for next best diagnostic steps; offering ‘Bias Mitigation’ by flagging potential cognitive errors such as anchoring and confirmation bias; enabling ‘Reassessment Prompting’ to trigger re-evaluation if a patient fails to respond to treatment; and allowing for ‘Specialty Tailoring’ to apply system-based diagnostic frameworks relevant to specific fields like neurology or cardiology. The DDX mode may leverage its knowledge base of medical conditions, symptom correlations, symptom clusters, and diagnostic criteria to generate a ranked or weighted list of possible diagnoses. Moreover, the generative AI model 90, when operating in DDX Mode, may suggest evidence-based diagnostic pathways by referencing established classification systems such as ICD-11 or DSM-5, incorporating analyses of functional medicine markers, and utilizing principles of naturopathic pattern recognition to inform its diagnostic suggestions. Furthermore, the DDX Mode can be configured to work in conjunction with other specialized modes, such as the Mechanism of Action (MOA) Mode to understand underlying pathological processes, or the Medical Doctor (MD) Writing Mode to help structure the diagnostic reasoning, generate summaries, or provide a differential diagnosis, for example. This mode may also be combined with the TS and/or the ND modes for deeper insight into pharmacologic, nutritional, or toxicological etiologies. This mode is typically activated with a command such as “Use DDX Mode” or a similar instruction, enabling healthcare professionals to utilize the analytical capabilities of the nutraceutical system 10 as a supportive tool in diagnostic decision-making.
43. Hospital Consult Mode (Consult Mode): This mode configures the generative AI model 90 to simulate a hospital-based specialty consultation process. The Consult Mode is designed to mirror real-world consult workflows where a specialist physician or practitioner provides expert recommendations, diagnostic input, or procedural guidance at the request of another healthcare provider managing a patient. The core functions of the Hospital Consult Mode include providing a ‘Structured Consult Response,’ which typically encompasses an Impression, Recommendations, and a Plan, similar to standard medical consultation reports; performing ‘Specialty Simulation’ by tailoring the tone, terminology, and content of its output to specific medical fields such as cardiology, infectious disease, neurology, or palliative care, based on user-defined parameters; offering ‘Ethical, Legal, and Safety Support’ by assisting in the analysis of complex cases that may involve end-of-life decisions, patient safety incidents, or legal and regulatory compliance issues; and providing ‘Collaborative Care Advice,’ which helps to clarify when the consulting service should assume full management of a patient's condition or when care should be shared between the referring and consulting providers. The operation of this mode may be customized using parameters such as the Service (e.g., Neurology), Urgency (e.g., Stat), Setting (e.g., Inpatient), and SharedCare status (e.g., Yes/No). The Hospital Consult Mode is designed to integrate and work seamlessly with other specialized modes of the nutraceutical system 10, such as the Differential Diagnosis Mode (DDX) for diagnostic input, the Medical Doctor (MD) Writing Mode for documentation, the Integrative Medicine Consultant (IMC) mode for holistic care planning, and the Toxicology & Safety Assessment (TS) mode for evaluating safety aspects. This mode is typically activated with a command such as “Use Consult Mode” or a similar user instruction. The generative AI model 90, operating in one or more modes disclosed herein, crafts responses designed to be detailed, engaging, and user-friendly, providing clear, concise, and accurate information. The generative AI model 90 can generate reports on natural compounds, explain their mechanism of action, advise on their integration with conventional treatments, or create detailed wellness plans. Thus, the generative AI model 90, implementing one of the modes described herein, provides a technical solution to the technical problem of generative AI hallucination and conversation drift.

In some embodiments, the generative AI model 90 may be configured to operate through predefined collections or integrations of its specialized modes to address more complex or multifaceted user queries, effectively creating a custom AI persona or assistant for specific domains. An exemplary embodiment of such an integration is a configuration referred to as “NutriChef AI,” which combines culinary expertise with nutritional science. In such a configuration, the generative AI model 90 may utilize a default combination, for example, of the Chef Mode and the Clinical Nutritionist Mode (ND), to provide functional recipes with detailed nutritional insights. Furthermore, this “NutriChef AI” configuration can be instructed to leverage other modes, such as the Food Scientist Mode (FS) for food chemistry and functional benefits, or the Clinical Nutrition Copywriter Mode (CN) for polished recipe presentations with health insights, or even the Article Style Mode (AS) for research-backed health articles. This illustrates the capability of the nutraceutical system 10 to provide multi-mode integration, for instance, by using the Food Scientist Mode (FS) in conjunction with the Clinical Nutrition Copywriter Mode (CN) to create a functional recipe for a specific health goal, thereby allowing for synergistic outputs that draw upon the distinct expertise of several individual modes to deliver a more comprehensive and tailored response to the user.

Furthermore, such combined mode configurations, for example the “NutriChef AI,” may not only leverage the inherent capabilities of their constituent modes but may also be designed to access or interact with other specialized modes of the generative AI model 90, or even distinct AI agent configurations within the nutraceutical system 10, as if those other modes or agents were specialized AI resources. This interaction can be conceptualized as one AI agent (representing the combined mode) querying or tasking another AI agent (representing a different specialized mode or a collection thereof) to obtain specific information or perform a sub-task. For instance, a combined mode like “NutriChef AI,” while primarily focused on culinary and nutritional aspects, could be configured to call upon the Pharmacognosy Research Mode (PR) or the Mechanism of Action Mode (MOA) as distinct AI agents to retrieve detailed pharmacological data or mechanistic insights about a specific herb or compound, which is then integrated into the final recipe or meal plan. This interaction may involve a sequential process where the output from one mode or AI agent serves as an input or refined prompt for another, allowing for a dynamic and multi-layered analysis to address complex user queries that span multiple domains of expertise within the nutraceutical system 10.

In a further exemplary embodiment of a combined mode configuration, referred to as “NutriChef AI,” the generative AI model 90 is specifically tailored to function as an assistant combining culinary expertise with nutritional science. The core functions of this “NutriChef AI” configuration include, but are not limited to: generating recipes and meal plans personalized to user-specified health goals (such as anti-inflammatory diets, sports nutrition, or mood balance); performing detailed nutritional analysis of meals, including macronutrient and micronutrient breakdowns and allergen detection; providing scientific insights into food, such as the mechanism of action of active compounds and their health benefits; suggesting symbiotic food pairings to enhance nutrient absorption and synergy; offering meal planning capabilities, potentially including shopping lists; focusing on gut health by suggesting probiotic and prebiotic-rich foods; providing eco-friendly food selection tips, such as highlighting sustainable or seasonal choices; and advising on cooking techniques designed to preserve nutrient integrity. This “NutriChef AI” configuration, while primarily leveraging modes such as Chef Mode, Clinical Nutritionist Mode (ND), Food Scientist Mode (FS), and Clinical Nutrition Copywriter Mode (CN), can also interact with the broader capabilities of the nutraceutical system 10.

For example, the “NutriChef AI” can be instructed to incorporate specific herbs or compounds identified by the Pharmacognosy Research Mode (PR) or Botanical Chemist Mode (BC) into its recipes. At the same time, the system concurrently provides pharmacological insights, such as pharmacokinetics or potential interactions for those ingredients, drawing from modes like PDK Mode or TS Mode. Such integration may be facilitated via internal API calls between different mode functionalities, through structured prompt interoperability, or through MCP integrations, allowing for a seamless blend of culinary advice with detailed pharmacological and nutritional data to create holistic health plans or educational content.

Another exemplary embodiment of an integrated, multi-modal agent configuration within the nutraceutical system 10 is the “Regenerative Bio-Integrator Agent (RBIA).” The RBIA configuration synthesizes complex biomedical data and generates actionable, evidence-based treatment strategies by fusing artificial intelligence with regenerative medicine, integrative health, and clinical nutrition. To achieve this, the RBIA configuration of the generative AI model 90 interacts with one or more (optionally, multi-modal) agent based on state-of-the-art AI models (e.g., via a protocol, such as MCP, on the network 26), which may include models such as GPT-4 or BioGPT, and is further configured to access and process information from biomedical databases, such as PubMed, Embase, and FoodData Central (such as via an API or other MCP connector). The processor 86 executing the generative AI mode 90 having the RBIA configuration therefore provides tailored insights for biomedical applications including but not limited to: stem cell activation, wound healing, metabolic optimization, and chronic disease management. The processor 86 executing the generative AI mode 90 configured with the RBIA mode leverages several of the specialized modes of the generative AI model 90, including, for example, the Nutritionist Mode (ND), the Wellness Life Coach Mode (WLC), the Integrative Medicine Consultant Mode (IMC) mode, and the AI-Enhanced Metabolic Pathway Mode (AMP).

Additionally, a combined mode configuration may include one or more mode exclusively available to the combined mode configuration. For example, the RBIA combined mode configuration may consist of a “Multiple Myeloma (MM) Mode.” The MM Mode configures the generative AI model 90 to provide specialized insights focused on Multiple Myeloma therapy, addressing areas such as integrative and conventional strategies, combination therapies, the use of cannabinoids, proteasome inhibitors, and naturally derived bioactive compounds (such as cerebrosides, lectins, sulfated polysaccharides, and saponins). When the MM Mode is active, for example within the RBIA configuration, the generative AI model 90 presents research-backed insights into mechanisms of action, synergy between treatments and conventional therapies, and potential integrative strategies to optimize patient outcomes in the context of Multiple Myeloma. The RBIA, through such combinations of modes including the specialized MM Mode, aims to provide tailored insights for applications ranging from stem cell activation and wound healing to metabolic optimization and chronic disease management, potentially incorporating real-time biometric tracking and dynamic metabolic pathway simulations to fine-tune dietary and therapeutic interventions for optimized chronic disease management. In one embodiment, the programmatically activate Multiple Myeloma (MM) Mode, the user 16 may provide input to the processor 58 to programmatically call a ‘set_mode’ function and pass in a string, such as MM as an argument, for example, as shown in pseudocode: ‘python agent.set_mode(“MM”)’.

In yet another embodiment, the nutraceutical system 10 having the processor 86 executing the generative AI model 90 enables processing a general user query and automatically determining an optimal combination or sequence of specialized modes to generate a comprehensive and relevant response. This process may involve the user 16 issuing a command such as “Choose the appropriate mode” or a similar instruction, or the nutraceutical system 10 may be configured to infer the need for multi-mode application based on the complexity or nature of the query. Upon such activation, the processor 86, executing the generative AI model 90, may analyze the user's query and select a plurality of the available specialized modes (e.g., from the modes described herein) and apply them, either sequentially or in a combined fashion, to produce the desired result. This intelligent mode selection and application allows the user to benefit from the collective expertise of multiple modes without needing to manually specify each one, thereby streamlining the interaction and enhancing the quality of the output.

The generative AI model 90 of the nutraceutical system 10 further incorporates significant “Innovation Dynamics” enhancements designed to overcome critical limitations in current pharmaceutical research, naturopathic practices, and overall healthcare models. These enhancements focus on three primary areas: Advanced AI-Driven Drug Interaction Analysis, Real-Time Biometric Tracking & Predictive Modeling, and Enhanced Naturopathic-Pharmaceutical Integration. These improvements aim to increase the precision, adaptability, and holistic nature of the analyses and recommendations provided by the nutraceutical system 10, thereby addressing key limitations such as slow manual research methodologies, siloed disciplinary approaches, and the lack of adaptive, AI-driven personalized treatments.

Specifically, these enhancements enable processor 86 to execute the generative AI model 90 and operate with further refined capabilities. In Advanced AI-Driven Drug Interaction Analysis, enhanced modes such as the Mechanism of Action (MOA), Pharmacodynamics & Pharmacokinetics (PDK), and Toxicology & Safety Assessment (TS) modes provide deeper insights into pharmacological pathways, drug ADME (absorption, distribution, metabolism, and elimination), and toxicity risk assessments. For Real-Time Biometric Tracking & Predictive Modeling, enhanced modes like the Nutritionist (ND), Wellness Life Coach (WLC), and Integrative Medicine Consultant (IMC) modes integrate capabilities for real-time nutrient bioavailability analysis, biometric-based wellness tracking (including stress and sleep data), and AI-driven chronic disease management with real-time interventions. Finally, in Enhanced Naturopathic-Pharmaceutical Integration, enhanced modes such as the Pharmacognosy Research (PR) and Phytopharmaceutical (PHYTO) modes extend bioactive compound discovery, AI-driven profiling of plant-based molecules, and AI-based botanical standardization. In this way, the generative AI model 90 of the nutraceutical system 10, with enhanced capabilities across various specialized operational modes, provides a solution to the technical problem of effectively integrating traditional medicine with advanced pharmaceutical research and overcoming limitations of siloed data and manual analysis which hinder and slow precision healthcare.

In one embodiment, the predetermined prompt modes may be included in the one or more AI prompt by the user 16 by including the activation command, the prompt identifier, and/or the numerical identifier in the natural language input into the input device 50 of the user system 14 and provided to the one or more AI prompt. In this way, the processor 86 executing the generative AI model 90 crafts the responses to be detailed, engaging, and user-friendly, providing clear, concise, and accurate information. The processor 86 executing the generative AI model 90 can generate reports on natural compounds, explaining their mechanism of action, advising on their integration with conventional treatments, or creating detailed wellness plans.

In one embodiment, processing data using a machine learning system (step 158) includes the processor 58 generating one or more AI prompt supplied to the generative AI model 90 executed by the processor 86 by providing one or more predetermined prompt to the generative AI model 90. For example, in one embodiment, the generative AI model 90 may be referred to as a Combo Pharma Integrator (e.g., a second agent) when provided with the following predetermined prompt having prompt modes to the user 16 where each predetermined prompt mode includes the prompt identifier (described as a “mode” and preceded by a numerical identifier and including an activation command):
“1. Combination Therapy Mode: In this role you are to act as a Combination Therapy Expert, lets call it Combination Therapy Expert a is a professional with specialized knowledge and skills in the development and application of combination therapies. These therapies involve using multiple drugs in concert to treat various diseases. a Combination Therapy Expert is a professional with comprehensive knowledge and skills in pharmacology, clinical practices, research methodologies, regulatory standards, and interdisciplinary collaboration. You are to play a pivotal role in the development and application of combination drug therapies, aiming to enhance treatment efficacy, reduce toxicity, and address unmet medical needs, particularly in complex diseases. Provide detailed responses in a style that Informs, teaches, or explains concepts; often used in instructional or academic contexts. Present the information in relative groups and cite references. Activated by “Use CT mode.”
“The expertise of such an individual would encompass several critical areas: • Understanding of Pharmacology and Drug Interactions: An expert in combination therapy must have a deep understanding of pharmacology, particularly how different drugs interact with each other and the body. This includes knowledge of pharmacodynamics (how drugs affect the body) and pharmacokinetics (how the body processes drugs). • Clinical Experience and Knowledge: They should be knowledgeable about clinical practices and the application of combination therapies in various medical contexts. This includes an understanding of diseases that benefit from combination therapy, such as cancer, HIV/AIDS, and certain cardiovascular conditions. • Research and Development Skills: An integral part of their role involves researching and developing new combination therapies. This requires the ability to design and conduct clinical trials to evaluate the efficacy, safety, and potential toxicity of drug combinations. • Insight into Drug Resistance: They must understand how combination therapies can be used to prevent or overcome drug resistance, a significant concern in conditions like infectious diseases and cancer. • Ethical and Regulatory Knowledge: Being well-versed in ethical considerations and regulatory guidelines related to drug development and patient treatment is essential. This includes understanding the approval processes for new therapies and ensuring patient safety in clinical trials. • Analytical and Problem-Solving Skills: The ability to analyze complex medical and scientific data is crucial. They should be capable of solving problems related to drug interactions, side effects, and individual patient responses to therapy. • Collaborative Skills: Combination therapy often involves interdisciplinary collaboration, so an expert in this field should work effectively with other healthcare professionals, including doctors, pharmacists, and researchers. • Continuous Learning and Adaptation: The field of pharmacology is rapidly evolving, so staying updated with the latest research, treatments, and technological advancements is vital. • Educational Background: A pharmacognosist usually holds an advanced degree (Ph.D. or Master's) in pharmacognosy, pharmaceutical sciences, medicinal chemistry, or a related field like botany or biochemistry. • Expertise in Natural Products Chemistry: They have extensive knowledge of the chemistry of natural products, including the structure, function, and biosynthesis of plant and animal-derived compounds. • Skill in Drug Discovery and Development: Pharmacognosists are skilled in drug discovery and development processes. This includes screening natural substances for biological activity, isolating and characterizing active compounds, and assessing their potential as drug candidates. • Experience in Analytical Techniques: They are proficient in various analytical techniques used for compound isolation and identification, such as chromatography (HPLC, GC-MS), spectroscopy (NMR, mass spectrometry), and bioassays. • Knowledge of Pharmacology and Toxicology: Understanding the pharmacological effects of natural compounds on the body and their potential toxicological risks is crucial in this role. • Botanical Knowledge: A thorough understanding of botany and ethnobotany is essential, as it helps identify and classify medicinal plants and other natural sources of therapeutic compounds. • Regulatory Knowledge: They must be familiar with regulatory requirements and procedures for new drug development, including preclinical and clinical testing, to ensure that potential drug candidates meet safety and efficacy standards. • Interdisciplinary Collaboration: Pharmacognosists often work in collaboration with other scientists, including biochemists, molecular biologists, and clinicians, to advance the drug development process. • Research and Development (R&D) Skills: They typically engage in R&D activities, contributing to the advancement of knowledge in the field and the development of new therapeutic agents.
“2. Pharmacognosist Mode: Act as a pharmacognosist who is also a specialist who combines knowledge of natural sciences and pharmaceutical sciences to discover, analyze, and develop new drugs from natural sources. You are a Pharmacognosist specializing in Natural Product Chemistry or a Natural Product Chemist who is very experienced in conducting literature reviews and analyze synergistic effects across botanical chemistry, marine compounds, natural product science, and analytical skills to correlate results to demonstrate synergistic effects to contribute significantly to the field of drug discovery and development from natural sources. Your expertise is crucial in transforming a natural compound into a viable single molecule drug candidate. You also have a skill in identifying natural compounds that have the potential to improve the efficacy of existing pharmaceuticals. Activate by “Use Pharma Mode.”
“This role requires a blend of skills and knowledge areas: • Deep Understanding of Natural Product Chemistry: This includes expertise in both botanical chemistry and marine compound chemistry. A strong background in organic chemistry, especially related to natural products, is crucial. • Research Skills in Pharmacognosy: The specialist should be proficient in pharmacognosy, the study of medicinal drugs obtained from plants or other natural sources. They should be able to understand, analyze, and interpret complex chemical compositions and biological activities of natural products. • Experience in Synergistic Analysis: The ability to assess and correlate synergistic effects among natural compounds is essential. This involves understanding how different compounds interact and enhance each other's effects, which is critical in the development of combination therapies or complex natural product formulations. • Literature Review and Meta-Analysis Skills: Proficiency in conducting comprehensive literature reviews, systematic reviews, and meta-analyses is necessary. This includes the ability to critically evaluate and synthesize a wide range of scientific studies and data. • Interdisciplinary Knowledge: A broad understanding of related disciplines, including biochemistry, molecular biology, and pharmacology, is important for integrating knowledge from different areas of natural product science. • Analytical and Critical Thinking: The ability to analyze diverse sets of data, identify patterns and correlations, and draw meaningful conclusions about the synergistic effects of natural products. • Academic and Research Credentials: Typically, a Ph.D. or an advanced degree in a related field is required, along with a track record of published research in reputable scientific journals. • Regulatory and Ethical Knowledge: Understanding the regulatory aspects of natural product research, including ethical considerations in the sourcing and use of botanical and marine compounds. • Communication Skills: The ability to clearly communicate complex scientific information and findings to a variety of audiences, both in writing and verbally.
“3. Combo Pharma Assistant Mode: Act as an assistant to a specialist with qualifications in pharmacognosy, natural product chemistry and Combination Therapy. Combo Pharma Assistant Mode is activated by “Use Assistant Mode” • Research Assistance: Helping in the collection, organization, and preliminary analysis of research data. This might involve conducting literature searches, summarizing research articles, and compiling data for analysis. • Laboratory Support: If the specialist works in a laboratory setting, the assistant might be involved in preparing samples, maintaining laboratory equipment, and performing basic experiments or tests under the guidance of the specialist. • Administrative Tasks: Handling routine administrative duties like scheduling meetings, managing correspondence, organizing files, and maintaining research documentation. This ensures that the specialist can focus more on their core research and analysis tasks. • Technical Assistance: Assisting with technical tasks, which could range from setting up and calibrating equipment to running computer simulations or using software for data analysis. • Drafting and Editing: Helping in drafting research papers, reports, and presentations. This may involve initial writing, proofreading, formatting according to journal guidelines, and preparing visual aids like graphs and tables. • Communication and Coordination: Acting as a point of contact for the research team, collaborators, and other stakeholders. This may include coordinating project activities, meetings, and communications. • Data Management: Assisting in the management and organization of research data, ensuring that it is stored securely and is easily accessible for analysis. • Compliance and Safety: Ensuring that the laboratory and research practices comply with safety standards and regulatory requirements. This might involve managing safety protocols, chemical inventories, and waste disposal. • Learning and Adaptation: Staying informed about the latest research techniques, laboratory equipment, and data analysis tools to provide effective support in a rapidly evolving field. • Ethical and Legal Compliance: Assisting in ensuring that all research activities comply with ethical guidelines and legal requirements, particularly in relation to natural product sourcing and experimentation.

“The assistant's role is crucial in supporting the specialist to achieve the research objectives efficiently and effectively. They provide a blend of administrative, technical, and research support, enabling the specialist to focus on complex analytical and conceptual aspects of their work. The specific duties of the assistant can vary depending on the specialist's focus area, the nature of the research projects, and the organizational setting.”

Here is a list of well-respected reference guides that pharmacists and physicians commonly use to prescribe drugs to patients is the “Physicians' Desk Reference” (PDR). This comprehensive drug reference provides detailed information on prescription drugs, including indications, dosages, side effects, interactions, and contraindications. The PDR is a trusted resource used by healthcare professionals to ensure safe and effective medication use. Other notable reference guides include: • British National Formulary (BNF): Widely used in the United Kingdom, the BNF provides concise information on prescribing, dispensing, and administering medications. • Drug Information Handbook: A widely used resource by Lexicomp, it offers detailed drug information and is known for being user-friendly, especially in a clinical setting. • The Merck Manual: Known for its detailed and comprehensive medical information, this manual covers a broad range of topics including drug prescribing. • AHFS Drug Information: Published by the American Society of Health System Pharmacists, this guide is known for its extensive coverage of drug information and is often used in hospitals. • Epocrates: This is a mobile application that provides drug information, including dosing, drug interactions, and insurance coverage. It is popular for its convenience and ease of use in clinical settings. • UpToDate: An evidence-based, physician-authored clinical decision support resource which is frequently updated with the latest drug information and prescribing guidelines.”

In one embodiment, the predetermined prompt modes may be included, e.g., independently, in multiples of the generative AI models 90. For example, a first generative AI model may include the predetermined prompt modes and a first instruction causing the first generative AI model to be the first agent (described above, e.g., as the NPM Pharma integrator) and a second generative AI model may include the predetermined prompt modes and a second instruction causing the second generative AI model to be the second agent (described above, e.g., as the Combo Pharma Integrator). The first instruction and the second instruction may, for example, indicate to the respective agent, which mode to operate under, or, in other embodiments, for example, may provide an additional ones of the one or more AI prompts.

In some embodiments, the predetermined prompts having the predetermined prompt modes, knowledge, and/or additional instructions may be provided to respective generative AI models via an uploaded document (e.g., by providing a document having the predetermined prompt with the predetermined prompt modes, knowledge, and/or instructions to the server system 22 executing the generative AI models). The processor 86 may provide the document to the generative AI model to use during execution, such as to a context window of the generative AI model 90.

In addition to the aforementioned modes, the processor 86 executing the generative AI model 90 adheres to the Food and Nutrition Style rules when creating illustrations, and all illustrations are presented on a pure white background unless otherwise specified, ensuring the visuals align with user expectations in graphic design. Further, the processor 86 executing the generative AI model 90 adheres to the Food and Nutrition Style rules for DALLE-3 when creating illustrations, ensuring the visuals align with its expertise in one or more of naturopathic medicine, traditional Chinese medicine, Ayurvedic medicine, pharmacology, pharmacognosy, organic chemistry, radiology, and digital technology.

In one embodiment, analyzing data to identify patterns, correlations, and synergistic effects (step 162) includes the processor 58 and/or the processor 86 executing a comparative analysis across retrieved studies to assess a therapeutic role of compounds, including antiviral, anti-inflammatory, and immunomodulatory effects. The comparative analysis may be, for example, a multi-vector comparison.

In one embodiment, analyzing data to identify patterns, correlations, and synergistic effects (step 162) includes the processor 86 executing the generative AI model 90 to analyze collected data by performing a multi-vector comparison of compounds across a plurality of property vectors including, for example, mechanisms of action, pharmacokinetics, and pharmacodynamics to identify potential synergistic interactions. In this way, the processor 86 executing the generative AI model 90 may match synergistic natural compounds to pharmaceuticals based on the MOA and published peer reviewed papers.

In one embodiment, analyzing data to identify patterns, correlations, and synergistic effects (step 162) includes the processor 86 executing the generative AI model 90 to identify patterns, correlations, and synergistic effects within the generated response. For example, the generated response having the processed data may be included in a second query presented to the generative AI model 90 (or a second one of a generative AI model 90) that, when executed by the processor 86, may provide patterns, correlations, and synergistic effects to the user 16 via the output device 54 of the user system 14.

In one embodiment, analyzing data to identify patterns, correlations, and synergistic effects (step 162) includes the processor 86 executing the generative AI model 90 receiving the processed data and analyzing the processed data against one or more data source, such as, peer-reviewed publications, the NIH, the WHO, and/or other international sources.

In one embodiment, analyzing data to identify patterns, correlations, and synergistic effects (step 162) includes the processor 86 executing the generative AI model 90 to analyze data related cannabinoid (and, optionally, other natural compounds) for therapeutic utilization.

In one embodiment, generating insights into roles of compounds in combination therapies (step 166) includes the processor 86 executing the generative AI model 90 to generate insights into the roles of compounds in combination therapies. For example, the generated response (from step 158 and/or step 162) may be provided in a third query presented to the generative AI model 90 (or a second one of a generative AI model 90) that, when executed by the processor 86, may provide insights into the roles of compounds (such as those presented by the user 16 in the inputs) in combination therapies via the output device 54 of the user system 14.

In one embodiment, generating insights into roles of compounds in combination therapies (step 166) may include generating a therapeutic intervention recommending alternative pharmaceuticals to use with natural compounds and/or recommending off-label uses of therapeutics, and/or the like, or a combination thereof.

In one embodiment, generating insights into roles of compounds in combination therapies (step 166) includes the processor 86 executing the generative AI model 90 to generate a report on identified compounds and may include, for example, summarized findings. For example, the processor 86 executing the generative AI model 90 may generate the report on the identified compounds in accessible language that may be understandable by the user 16 (who may not be a medical professional). In this way, the processor 86 is able to transform data provided to the user 16 into a format (e.g., syntax and diction) that the user 16 would otherwise be unable to process or understand. In one embodiment, a report may be generated for each of the identified compounds.

In one embodiment, generating insights into roles of compounds in combination therapies (step 166) includes providing a response in one or more response language. The one or more response language may be different from a language used by the user 16 when inputting the user query. Further, the one or more user query language and the one or more response language may be different from a language of the knowledgebase data used as a basis for the generated response.

In one embodiment, generating insights into roles of compounds in combination therapies (step 166) further includes the processor 86 executing the generative AI model 90 generating the report to format the report with one or more of: body citations, references, links to published papers, clinical evidence, and/or associated pharmacokinetics and drug interactions. In this way, generation of hallucinations by the generative AI model 90 may be minimized.

In one embodiment, generating insights into roles of compounds in combination therapies (step 166) further includes the processor 86 executing the generative AI model 90 to generate a report detailing identified synergistic interactions to offer a therapeutic benefit for the medical condition.

In one exemplary implementation of the generative AI model 90, the processor 86 executing the generative AI model 90 receives input from the user 16 (via the input device 50 of the user system 14) for a specific disease, such as cancer, and a compound, such as curcumin, along with a category of natural compounds, such as antioxidants. The processor 86 executing the generative AI model 90 then searches for and analyzes the (optionally, 10) most researched antioxidants that can be blended with curcumin to create synergistic blends for cancer treatment. The processor 86 executing the generative AI model 90 reviews clinical evidence from relevant sources and generates a report on the identified natural compounds, their sources, pharmacokinetics, and potential drug interactions. The processor 86 executing the generative AI model 90 summarizes the findings and draws a conclusion on the efficacy of the identified natural compounds in combination with curcumin for cancer treatment.

Further, in some embodiments, the processor 86 executing the generative AI model 90 may provide a predictive analysis, a risk assessment, and a real-time therapy adjustment for the user 16 and/or a patient. In this way, the processor 86, by analyzing data to identify patterns, correlations, and synergistic effects (step 162), not only meets the urgent need for more effective therapies but also ensures high standards of safety and regulatory compliance, making the nutraceutical system 10 a significant advancement in the field of medical treatment.

In one embodiment, providing the predictive analysis, the risk assessment, and the real-time therapy adjustment in an integrated generated response ensures that safety aspects of identified combination therapies are included in the generated response as well as identified as a consideration for the user 16. In one embodiment, the processor 86 of the nutraceutical system 10 efficiently identifies, analyzes, and matches natural compounds with pharmaceuticals based on the Mechanism of Action (MOA), pharmacokinetics, and pharmacodynamics of the compounds and pharmaceuticals. Further the processor 86 of the nutraceutical system 10 efficiently addresses drug resistance, toxicity, and efficacy issues in treatment. In this way, the nutraceutical system 10 is provided with a robust framework for evaluating the safety of natural compounds used in combination with pharmaceuticals, while simultaneously emphasizing regulatory challenges and health risks of the combinations.

In another exemplary implementation of the generative AI model 90, the processor 86 executing the generative AI model 90 receives input for a specific disease, such as diabetes, and a compound, such as metformin, along with a category of natural compounds, such as herbs or polysaccharides (with sources of the compound cited). The processor 86 executing the generative AI model 90 then searches for and analyzes the 10 most researched herbs that can be blended with metformin to create synergistic blends for diabetes treatment. The processor 86 executing the generative AI model 90 reviews clinical evidence from relevant sources and generates a report on the identified natural compounds, their sources, pharmacokinetics, and potential drug interactions. The processor 86 executing the generative AI model 90 summarizes the findings and draws a conclusion on the efficacy of the identified natural compounds in combination with metformin for diabetes treatment.

In another exemplary implementation of the generative AI model 90, the processor 86 executing the generative AI model 90 may also be instructed to research and analyze (e.g., by the user 16 or other generative AI model 90) combinations of natural compounds with pharmaceutical drugs to identify potential synergistic effects. For instance, the processor 86 executing the generative AI model 90 could analyze the combination of the natural compound turmeric with the pharmaceutical drug tamoxifen for breast cancer treatment. This process involves the processor 86 executing the generative AI model 90 reviewing existing clinical studies, identifying pharmacokinetic interactions, and summarizing the potential benefits and risks of the combination therapy.

In another exemplary implementation of the generative AI model 90, the processor 86 executing the generative AI model 90 may also be instructed to research and analyze (e.g., by the user 16 or other generative AI model 90) combinations of natural compounds with pharmaceutical drugs to identify potential synergistic effects while taking into account the private medical information of the user 16. In this way, the combination therapy result can be personalized to the user 16 and/or used as a standard treatment.

Referring now to FIG. 5, shown therein is a screenshot of an exemplary embodiment of a user interface 200 constructed in accordance with the present disclosure. The user interface 200 may provide the user 16 a list of prompts selectable via interaction with a first tab 204 and a prompt configuration pane 206 selectable via interaction with a second tab 208. In one embodiment, the user 16 may provide a search input to an input 212. The processor 86 may receive the search input from the input 212 and query the list of prompts such that only prompts having or associated with the search input are provided in the list of prompts.

In one embodiment, the user 16 may edit a prompt configuration via the second tab 208 having the prompt configuration pane 206. The prompt configuration pane 206 may be provided with a plurality of inputs operable to, when received by the processor 86, be used to generate a prompt algorithm to the one or more generative AI model 90 and to save the prompt algorithm, for example, in the memory 82 such as in the database 94. The prompt configuration pane 206 may include, for example, a prompt query 216, a response configuration input 218 (for example, to allow the user 16 to select a particular analysis method such as pharmacokinetics and/or pharmacodynamics) and a generated response output 220. In one embodiment, the prompt algorithm may be provided a prompt name via name input 222. The generated response output 220 may display a response from the generative AI model 90 executing the prompt algorithm. When more than one generative AI model 90 is utilized (based on one or more generative AI model selector 238 discussed below), the response output 220 may display a response from each of the one or more generative AI models 90, for example, side-by-side.

In one embodiment, the user interface 200 is provided with a prompt toggle 224 for each prompt mode selector 226. As shown in FIG. 5, the user interface 200 is provided with a first prompt toggle 224a, a second prompt toggle 224b, and a third prompt toggle 224c operable to identify wither a first prompt mode 226a, a second prompt mode 226b, or a third prompt mode 226c, respectively, is utilized (or included) in the prompt algorithm.

In one embodiment, the user interface 200 is further provided with one or more bioactive component input 230, shown in FIG. 5 as a first bioactive component input 230a corresponding, for example, to a particular compound, a second bioactive component input 230b corresponding, for example, to a particular botanical, and a third bioactive component input 230c corresponding, for example, to a particular drug. Each bioactive component input 230 may be associated with a respective component inclusion indicator 232 operable to, cause the processor 86 to include the associated bioactive component input 230 in the prompt algorithm.

In one embodiment, the user interface 200 is further provided with one or more prompt configuration input 234 operable to, upon selection by the user 16, cause the processor 86 to include a predetermined configuration input in the prompt algorithm. For example, the one or more prompt configuration inputs 234 may include: a first prompt configuration input 234a operable to, upon selection, cause the processor 86 to include a first predetermined configuration input corresponding to a list of synergies in the prompt algorithm, a second prompt configuration input 234b operable to, upon selection, cause the processor 86 to include a second predetermined configuration input corresponding to a command to “Match the MOA” in the prompt algorithm, and a third prompt configuration input 234c operable to, upon selection, cause the processor 86 to include a third predetermined configuration input corresponding to a command to “find similar compounds” in the prompt algorithm.

In one embodiment, the user interface 200 is further provided with one or more generative AI model selector 238 operable to, upon selection by the user 16, cause the processor to transmit the prompt algorithm to one or more generative AI model 90 corresponding to the selected generative AI model selectors 238. For example, if a first generative AI model selector 238a was selected by the user 16, the processor 86, when executing the particular prompt, may transmit the prompt algorithm to the first generative AI model selector 238a.

In one embodiment, the user interface 200 is further provided with one or more formatting option 240 and one or more export option input 242. The processor 86 may format an exported prompt algorithm based on the one or more formatting option 240 and may export the exported prompt algorithm into a file having a file type based on the export option input 242 (e.g., a file type of, for example, a txt file, a PDF file, a DOCX file, and the like).

In one embodiment, the user interface 200 is further provided with one or more file import option 246. Upon selection of the one or more file import option 246, the processor 86 may, for example, present the user 16 with a file upload dialog operable to allow the user 16 to select a file that is accessible within for example, the user system 14 and/or the server system 22. In some embodiments, the file may be accessible via the network 26 and the user 16 may input, for example, a URL or other identifier for the file to upload. In some embodiments, the user 16 may drag and drop a file into the prompt configuration pane 206 of the user interface 200 (or onto the one or more file import option 246, for example) to cause the processor 86 to upload the dropped file. The uploaded files may be shown in a file list 248 having each uploaded file 250 and a delete button 252 operable to, upon selection, cause the processor 86 to remove the uploaded file 250 from one or more of: the file list 248, the prompt algorithm, the memory 82, the server system 22, the user system 14, and/or the like. In one embodiment, the file import option 246 may further allow the user 16 to insert, upload, or otherwise associate a particular hologram with the prompt algorithm thereby causing the generative AI model 90 to generate a personalized medical plan for the user 16 by taking data from the patient history (which is included in the hologram) and matching the patient history to different kinds and type of medicines.

Furthermore, in connection with the file import option 246 and the management of uploaded files 250, including potentially sensitive data such as a user's hologram or patient history, the nutraceutical system 10 may incorporate Web3 technologies, including blockchain. Such technologies can provide users 16 with enhanced control over their data, secure mechanisms for data sharing and consent management, and an immutable record of data provenance and access. For example, the user 16 uploading the hologram or other personal medical data could have ownership and sharing preferences of the user 16 recorded on a blockchain, ensuring that the use of such data by the generative AI model 90 adheres to auditable, user-defined permissions, thereby supporting data privacy and user trust within the framework of this disclosure.

In one embodiment, the user interface 200 is further provided with a save button 260 operable to receive an input from the user 16 and cause the processor 86 to save the prompt algorithm to the memory 82, for example, in the database 94.

These exemplary implementations demonstrate how the method and system disclosed herein (such as the nutraceutical system 10 constructed in accordance with the present disclosure) can be used to improve the efficacy of traditional medicine in treating various diseases, providing a novel approach to the development of combination therapies.

### ILLUSTRATIVE IMPLEMENTATIONS

The following is a non-limiting list of illustrative implementations in accordance with the present disclosure:

Illustrative Implementation 1. A computer-implemented method for identifying synergistic natural compounds for combination therapy in medicine, comprising:
receiving input for a specific disease, compound, and category of natural compounds;
utilizing an AI agent to search for and analyze the 10 most researched natural compounds from the specified category that can be blended with the input compound;
reviewing clinical evidence from peer-reviewed publications, the NIH, and other international sources;
generating a report for each identified natural compound, including its sources, clinical evidence supporting its positive effects on the disease, pharmacokinetics, and potential drug interactions; and
summarizing the findings and drawing a conclusion.

Illustrative Implementation 2. The method of Illustrative Implementation 1, further comprising formatting the report to include body, citations, references, links to published papers, and associated pharmacokinetics and drug interactions, if any.

Illustrative Implementation 3. The method of Illustrative Implementation 1, wherein the AI agent has a background in biotechnology, life sciences, and computer science.

Illustrative Implementation 4. The method of Illustrative Implementation 1, wherein the identified natural compounds are used to improve the efficacy of traditional medicine in treating the specified disease.

Illustrative Implementation 5. A system for identifying synergistic natural compounds for combination therapy in medicine, comprising:
an input module for receiving input on a specific disease, compound, and category of natural compounds;
an AI agent configured to search for and analyze the 10 most researched natural compounds from the specified category that can be blended with the input compound;
a review module for analyzing clinical evidence from peer-reviewed publications, the NIH, and other international sources;
a report generation module for creating a report on each identified natural compound, including its sources, clinical evidence supporting its positive effects on the disease, pharmacokinetics, and potential drug interactions; and
a summarization module for summarizing the findings and drawing a conclusion.

Illustrative Implementation 6. A method for identifying potential compounds for combination therapies, comprising:
collecting data from multiple studies on therapeutic effects of compounds;
processing data using an AI-driven tool with machine learning algorithms;
analyzing data to identify patterns, correlations, and synergistic effects; and
generating insights into roles of compounds in combination therapies.

Illustrative Implementation 7. The method of Illustrative Implementation 6, specifically applied to cannabinoids and their interactions with other pharmacological agents or natural extracts.

Illustrative Implementation 8. The method of Illustrative Implementations 6 or 7, further including comparative analysis across studies to assess therapeutic roles of compounds, including antiviral, anti-inflammatory, and immunomodulatory effects.

Illustrative Implementation 9. A system for implementing the method of Illustrative Implementations 6-8, comprising:
an AI tool for handling large datasets;
a database of research studies and clinical data on compounds;
a user interface for data input and retrieval of analysis results; and
machine learning algorithms for identifying synergistic effects in combination therapies.

Illustrative Implementation 10. The system of Illustrative Implementation 9, where the AI tool is further specialized in analyzing cannabinoid-related data for therapeutic use.

Illustrative Implementation 11. A computer-implemented method for identifying synergistic natural compounds for combination therapy, involving:
receiving input for a specific disease and compound;
utilizing an AI agent to search and analyze natural compounds;
reviewing clinical evidence;
generating a report on identified compounds; and
summarizing findings.

Illustrative Implementation 12. The method of Illustrative Implementation 11, further comprising formatting the report with body citations, references, links to published papers, and associated pharmacokinetics and drug interactions.

Illustrative Implementation 13. The method of Illustrative Implementation 11, where the AI agent possesses expertise in biotechnology, life sciences, and computer science.

Illustrative Implementation 14. The method of Illustrative Implementation 11, wherein the identified natural compounds are intended to enhance the efficacy of traditional medicine for treating the specified disease.

Illustrative Implementation 15. A system for identifying synergistic natural compounds for combination therapy, comprising:
an input module for receiving disease and compound-specific information;
an AI agent configured to analyze natural compounds;
a review module for analyzing clinical evidence;
a report generation module; and
a summarization module.

Illustrative Implementation 16. A system, comprising: a processor; and a memory, comprising a non-transitory processor-readable medium, storing processor-executable instructions and a generative AI agent, that when executed by the processor, cause the processor to:
collect data from a plurality of related studies on therapeutic effects of particular compounds;
configure a generative AI model with one or more specialized modes selected from a plurality of predefined modes, wherein each specialized mode directs the generative AI agent to apply a distinct set of analytical rules and provides access to domain-specific knowledge bases;
analyze, by the configured generative AI model, the collected data based on the distinct set of analytical rules by performing a multi-vector comparison of the particular compounds across a plurality of property vectors including mechanisms of action, pharmacokinetics, and pharmacodynamics to identify potential synergistic interactions;
generate a report detailing identified synergistic interactions to offer a therapeutic benefit based on the comparison of the particular compounds.

Illustrative Implementation 17. The system of Illustrative Implementation 16, wherein the instruction to collect data from the plurality of related studies on therapeutic effects of the particular compounds further includes:
retrieving one or more study related to a user query; and
provide at least part of the one or more study to a context window of the generative AI model.

Illustrative Implementation 18. The system of Illustrative Implementation 16, wherein the memory further includes one or more database storing a plurality of studies, and wherein the instructions to collect data from the plurality of related studies on therapeutic effects of the particular compounds further includes:
receive one or more input from the user as a user query;
process the user query into a database query of one or more database in the memory; and
retrieve the plurality of related studies from the one or more database in the memory by identifying a set of the plurality of studies having a similarity to the user query.

Illustrative Implementation 19. The system of Illustrative Implementation 18, wherein the one or more database is a vector database.

Illustrative Implementation 20. The system of Illustrative Implementation 18, wherein retrieving the plurality of related studies further includes retrieving the plurality of related studies from a third-party service accessible via an API.

Illustrative Implementation 21. The system of Illustrative Implementation 16, wherein the memory further stores processor-executable instructions causing the processor to: receive one or more input from the user as a user query.

Illustrative Implementation 22. The system of Illustrative Implementation 21, wherein the user query includes one or more request having information regarding one or more of: a disease, a compound, and a natural compound.

Illustrative Implementation 23. The system of Illustrative Implementation 21, wherein the instruction to collect data from the plurality of related studies further includes: vectorizing the user query using the generative AI agent to determine the plurality of related studies.

Illustrative Implementation 24. The system of Illustrative Implementation 16, wherein the instruction to collect data from the plurality of related studies further includes: collecting data from the plurality of related studies on therapeutic effects of cannabinoids and cannabinoid interaction with other pharmaceutical agents or natural extracts.

Illustrative Implementation 25. The system of Illustrative Implementation 16, wherein processing data using a machine learning system further includes the processor executing the generative AI agent to process the data.

Illustrative Implementation 26. The system of Illustrative Implementation 25, wherein processing data using a machine learning system further includes generating one or more AI prompt supplied to the generative AI agent.

Illustrative Implementation 27. The system of Illustrative Implementation 26, wherein the one or more AI prompt may be a natural-style prompt.

Illustrative Implementation 28. The system of Illustrative Implementation 27, wherein the natural-style prompt is a natural language prompt.

Illustrative Implementation 29. The system of Illustrative Implementation 27, wherein the natural-style prompt is a natural speech prompt.

Illustrative Implementation 30. The system of Illustrative Implementation 16, wherein the memory further stores one or more AI prompt template having one or more prompt placeholders, and wherein the processor-executable instructions further cause the processor to: receive one or more input from the user indicative of an input to the one or more prompt placeholders.

Illustrative Implementation 31. The system of Illustrative Implementation 30, wherein the memory further stores processor-executable instructions that further cause the processor to: generate an AI prompt based on the one or more inputs from the user and the AI prompt template.

Illustrative Implementation 32. The system of Illustrative Implementation 16, wherein the instructions to generate the report detailing identified synergistic interactions to offer a therapeutic benefit for the medical condition further includes instructions to: generate a therapeutic intervention recommending at least one of: alternative pharmaceuticals to use with natural compounds; and off-label uses of therapeutics.

Illustrative Implementation 33. The system of Illustrative Implementation 16, wherein the instructions to generate the report detailing identified synergistic interactions to offer a therapeutic benefit for the medical condition further includes instructions to: generate the report to be understandable by a user, by:
determining a target accessible language for the user based on a received user prompt or a user account associated with the user; and
generating the report having the target accessible language.

The foregoing description provides illustration and description, but is not intended to be exhaustive or to limit the inventive concepts to the precise form disclosed. Modifications and variations are possible in light of the above teachings or may be acquired from practice of the methodologies set forth in the present disclosure.

From the above description, it is clear that the inventive concept(s) disclosed herein are well adapted to carry out the objects and to attain the advantages mentioned herein, as well as those inherent in the inventive concept(s) disclosed herein. While the embodiments of the inventive concept(s) disclosed herein have been described for purposes of this disclosure, it will be understood that numerous changes may be made and readily suggested to those skilled in the art which are accomplished within the scope and spirit of the inventive concept(s) disclosed herein.

Even though particular combinations of features are recited in the claims and/or disclosed in the specification, these combinations are not intended to limit the disclosure. In fact, many of these features may be combined in ways not specifically recited in the claims and/or disclosed in the specification. Although each dependent claim listed below may directly depend on only one other claim, the disclosure includes each dependent claim in combination with every other claim in the claim set.

No element, act, or instruction used in the present application should be construed as critical or essential to the invention unless explicitly described as such outside of the preferred embodiment. Further, the phrase “based on” is intended to mean “based, at least in part, on” unless explicitly stated otherwise.

What is claimed is:

1. A system, comprising:
a processor; and
a memory, comprising a non-transitory processor-readable medium, storing processor-executable instructions and a generative AI agent, that when executed by the processor, cause the processor to:
collect data from a plurality of related studies on therapeutic effects of particular compounds;
configure a generative AI model with one or more specialized modes selected from a plurality of predefined modes, wherein each specialized mode directs the generative AI agent to apply a distinct set of analytical rules and provides access to domain-specific knowledge bases;
analyze, by the configured generative AI model, the collected data based on the distinct set of analytical rules by performing a multi-vector comparison of the particular compounds across a plurality of property vectors including mechanisms of action, pharmacokinetics, and pharmacodynamics to identify potential synergistic interactions; and
generate a report detailing identified synergistic interactions to offer a therapeutic benefit based on the comparison of the particular compounds.

2. The system of claim 1, wherein the instruction to collect data from the plurality of related studies on therapeutic effects of the particular compounds further includes:
retrieving one or more study related to a user query; and
provide at least part of the one or more study to a context window of the generative AI model.

3. The system of claim 1, wherein the memory further includes one or more database storing a plurality of studies, and wherein the instructions to collect data from the plurality of related studies on therapeutic effects of the particular compounds further includes:
receive one or more input from the user as a user query;
process the user query into a database query of one or more database in the memory; and
retrieve the plurality of related studies from the one or more database in the memory by identifying a set of the plurality of studies having a similarity to the user query.

4. The system of claim 3, wherein the one or more database is a vector database.

5. The system of claim 3, wherein retrieving the plurality of related studies further includes retrieving the plurality of related studies from a third-party service accessible via an API.

6. The system of claim 1, wherein the memory further stores processor-executable instructions causing the processor to:
receive one or more input from the user as a user query.

7. The system of claim 6, wherein the user query includes one or more request having information regarding one or more of: a disease, a compound, and a natural compound.

8. The system of claim 6, wherein the instruction to collect data from the plurality of related studies further includes:
vectorizing the user query using the generative AI agent to determine the plurality of related studies.

9. The system of claim 1, wherein the instruction to collect data from the plurality of related studies further includes:
collecting data from the plurality of related studies on therapeutic effects of cannabinoids and cannabinoid interaction with other pharmaceutical agents or natural extracts.

10. The system of claim 1, wherein processing data using a machine learning system further includes the processor executing the generative AI agent to process the data.

11. The system of claim 10, wherein processing data using a machine learning system further includes generating one or more AI prompt supplied to the generative AI agent.

12. The system of claim 11, wherein the one or more AI prompt may be a natural-style prompt.

13. The system of claim 12, wherein the natural-style prompt is a natural language prompt.

14. The system of claim 12, wherein the natural-style prompt is a natural speech prompt.

15. The system of claim 1, wherein the memory further stores one or more AI prompt template having one or more prompt placeholders, and wherein the processor-executable instructions further cause the processor to:
receive one or more input from the user indicative of an input to the one or more prompt placeholders.

16. The system of claim 15, wherein the memory further stores processor-executable instructions that further cause the processor to:
generate an AI prompt based on the one or more inputs from the user and the AI prompt template.

17. The system of claim 1, wherein the instructions to generate the report detailing identified synergistic interactions to offer a therapeutic benefit for the medical condition further includes instructions to:
generate a therapeutic intervention recommending at least one of: alternative pharmaceuticals to use with natural compounds; and off-label uses of therapeutics.

18. The system of claim 1, wherein the instructions to generate the report detailing identified synergistic interactions to offer a therapeutic benefit for the medical condition further includes instructions to:
generate the report to be understandable by a user, by:
determining a target accessible language for the user based on a received user prompt or a user account associated with the user; and
generating the report having the target accessible language.
